(19) United States
(12) Patent Application Publication
(10) Pub. No.: US 2025/0134400 A1
(43) Pub. Date: May 1, 2025

(54) Pump System For Portable Non-Invasive Blood Pressure Monitoring System

(71) Applicant: Becton, Dickinson and Company, Franklin Lakes, NJ (US)

(72) Inventors: Huy Thanh Vu, Westminster, CA (US); Greg Joseph Dugan, Corona, CA (US); Abdulkader Sudam, Dana Point, CA (US); Ilja Guelen, Ravenstein (NL); Trent Gardner Callcott, Newport Beach, CA (US); Brennen Neal Wears, Mission Viejo, CA (US)

(21) Appl. No.: 18/933,947

(22) Filed: Oct. 31, 2024

Related U.S. Application Data
(60) Provisional application No. 63/594,897, filed on Oct. 31, 2023.
(60) Provisional application No. 63/645,578, filed on May 10, 2024.

(51) Int. Cl. A61B 5/022; A61B 5/021

(52) U.S. Cl. CPC A61B 5/02241; A61B 5/02141

(57) ABSTRACT

Disclosed herein are various pump systems for a portable blood pressure monitoring system. One particular pump system includes: a pump including an inlet and an outlet; inlet pressure chambers connected to the inlet, where the inlet pressure chambers are connected in series to such that a last inlet pressure chamber is connected to the inlet of the pump and a beginning inlet pressure chamber is connected to outside air; outlet pressure chambers connected to the outlet, where each of the outlet pressure chambers are connected in series such that a beginning outlet pressure chamber is connected to the outlet of the pump and a last outlet pressure chamber is connected to a port.

### CROSS-REFERENCE TO RELATED APPLICATIONS

This application claims priority to and the benefit of U.S. Provisional Application No. 63/594,897, filed Oct. 31, 2023, and U.S. Provisional Application No. 63/645,578, filed on May 10, 2024, the disclosures of which are hereby incorporated herein by reference in their entireties.

### FIELD

The present disclosed technology generally relates a pump system for a portable non-invasive blood pressure monitoring system.

### BACKGROUND

Continuous noninvasive blood pressure monitors enable real-time measurement of blood pressure waves and derived hemodynamic parameters. Multiple techniques can be utilized including the volume clamp method.

Volume clamp method measures arterial blood pressure at an extremity (e.g., finger) utilizing an inflatable cuff, a light source (e.g., light emitting diode (LED)), and light sensor. The pressure in the cuff is adjusted to keep the diameter of the artery constant (the unloaded state), in which the diameter is determined via the light source and light sensor. The pressure within the inflatable cuff represents the arterial pressure of the finger artery. A pressure pump supplies the pressure to the inflatable cuff.

### SUMMARY

In some aspects, the techniques described herein relate to a pump system including: a tortuous path; and a pump which is connected to an outlet of the tortuous path, wherein an inlet of the tortuous path is configured to receive outside air such that the received air travels through the tortuous path prior to entering the pump.

In some aspects, the techniques described herein relate to a pump system, wherein the tortuous path has no moving element.

In some aspects, the techniques described herein relate to a pump system, further including a volume accumulator attached to the pump which has a volume equal to a stroke volume of the pump.

In some aspects, the techniques described herein relate to a pump system, further including an orifice attached to the pump which receives air from the volume accumulator and wherein a diameter of the orifice is 0.1-times the stroke volume of the pump and the volume of the accumulator.

In some aspects, the techniques described herein relate to a pump system, wherein the pump includes one or more active damping units which measure pressure of volume ripples from the pump and control a flow of air.

In some aspects, the techniques described herein relate to a pump system, wherein the one or more active damping units includes a bellow or a poppet.

In some aspects, the techniques described herein relate to a pump system, wherein the tortuous path is elongated and snakes back and forth laterally.

In some aspects, the techniques described herein relate to a pump system, wherein the tortuous path has a circular cross-section with a diameter and a path length.

In some aspects, the techniques described herein relate to a pump system, wherein the path length of the tortuous path is 150-times to 200-times longer than a diameter of the tortuous path.

In some aspects, the techniques described herein relate to a pump system, wherein the path length is between 18 inches and 30 inches.

In some aspects, the techniques described herein relate to a pump system, further including a printed circuit board which controls the pump.

In some aspects, the techniques described herein relate to a pump system, further including a battery which powers the printed circuit board.

In some aspects, the techniques described herein relate to a pump system, further including a housing and a baseplate which cooperate to house the pump, the printed circuit board, and the battery.

In some aspects, the techniques described herein relate to a non-invasive blood pressure monitoring system including: a pump system including: a tortuous path; and a pump which is connected to an outlet of the tortuous path, wherein an inlet of the tortuous path is configured to receive outside air such that the air travels through the tortuous path prior to entering the pump, and a cuff including a bladder connected to an output of the pump system, wherein the bladder is configured to apply pressure to non-invasively measure blood pressure.

In some aspects, the techniques described herein relate to a method of damping pressure pulsations from a pump, the method including: providing air through an inlet of a tortuous path; and pumping the provided air by a pump connected to an outlet of the tortuous path, wherein the inlet of the tortuous path is configured to receive outside air such that the air travels through the tortuous path prior to entering the pump.

In some aspects, the techniques described herein relate to a method, wherein the inlet of the tortuous path provides reduction of noise created by the pump by having no moving element within the tortuous path.

In some aspects, the techniques described herein relate to a method, wherein the tortuous path has no moving element.

In some aspects, the techniques described herein relate to a method, wherein the tortuous path is elongated and snakes back and forth laterally.

In some aspects, the techniques described herein relate to a method, wherein the tortuous path has a circular cross-section with a diameter and a path length.

In some aspects, the techniques described herein relate to a method, wherein the path length of the tortuous path is 150-times to 200-times longer than a diameter of the tortuous path.

In some aspects, the techniques described herein relate to a pump system including: a pump including an inlet and an outlet; inlet pressure chambers connected to the inlet, wherein the inlet pressure chambers are connected in series to such that a last inlet pressure chamber is connected to the inlet of the pump and a beginning inlet pressure chamber is connected to outside air; outlet pressure chambers connected to the outlet, wherein each of the outlet pressure chambers are connected in series such that a beginning outlet pressure chamber is connected to the outlet of the pump and a last outlet pressure chamber is connected to a port.

In some aspects, the techniques described herein relate to a pump system, wherein the inlet pressure chambers are connected in series through one or more passages.

In some aspects, the techniques described herein relate to a pump system, wherein each of the one or more passages are formed by a first opening which includes an orifice, a second opening, and a channel, and wherein air is sucked from a first inlet pressure chamber through the orifice, through the channel, through the second opening, and into a second inlet pressure chamber.

In some aspects, the techniques described herein relate to a pump system, wherein the orifice is a thin disk material including a small hole.

In some aspects, the techniques described herein relate to a pump system, wherein the first opening and the second opening are formed in a gasket and the thin disk material of the orifice is thinner than the gasket.

In some aspects, the techniques described herein relate to a pump system, wherein each of the one or more passages are formed by a first opening, a second opening which includes an orifice, and a channel, wherein air is pushed through a first inlet pressure chamber, through the first opening, through the channel, through the orifice, and into a second inlet pressure chamber.

In some aspects, the techniques described herein relate to a pump system, wherein each of the one or more passages are formed by a first opening which includes a first orifice, a second opening which includes a second orifice, and a channel, wherein air is pushed through a first inlet pressure chamber, through the first orifice, through the channel, through the second orifice, and into a second inlet pressure chamber.

In some aspects, the techniques described herein relate to a pump system, wherein the inlet pressure chambers include three pressure chambers.

In some aspects, the techniques described herein relate to a pump system, wherein the outlet pressure chambers are connected in series through one or more passages.

In some aspects, the techniques described herein relate to a pump system, wherein each of the one or more passages are formed by a first opening which includes an orifice, a second opening, and a channel, and wherein air is pushed through a first outlet pressure chamber, through the orifice, through the channel, through the second opening, and into a second outlet pressure chamber.

In some aspects, the techniques described herein relate to a pump system, wherein the orifice is a thin disk material including a small hole.

In some aspects, the techniques described herein relate to a pump system, wherein the first opening and the second opening are formed in a gasket and the thin disk material of the orifice is thinner than the gasket.

In some aspects, the techniques described herein relate to a pump system, wherein each of the one or more passages are formed by a first opening, a second opening which includes an orifice, and a channel, wherein air is pushed through a first outlet pressure chamber, through the first opening, through the channel, through the orifice, and into a second outlet pressure chamber.

In some aspects, the techniques described herein relate to a pump system, wherein each of the one or more passages are formed by a first opening which includes a first orifice, a second opening which includes a second orifice, and a channel, wherein air is pushed through a first outlet pressure chamber, through the first orifice, through the channel, through the second orifice, and into a second outlet pressure chamber.

In some aspects, the techniques described herein relate to a pump system, wherein the outlet pressure chambers include four pressure chambers.

In some aspects, the techniques described herein relate to a pump system, wherein the last outlet pressure chamber is connected to the port through a piezoelectric valve.

In some aspects, the techniques described herein relate to a pump system, wherein the piezoelectric valve is switchable such that, in a first setting, the last outlet pressure chamber is connected to the port and, in a second setting, both the last outlet pressure chamber and the port are connected to outside air.

In some aspects, the techniques described herein relate to a pump system, further including a manifold including the inlet pressure chambers and the outlet pressure chambers and a printed circuit board including a first pressure transducer and a second pressure transducer, wherein the printed circuit board is mounted on the manifold with the piezoelectric valve positioned between the printed circuit board and the manifold, and wherein the piezoelectric valve is positioned between the first pressure transducer and the second pressure transducer.

In some aspects, the techniques described herein relate to a pump system, wherein the first pressure transducer is configured to measure the pressure at the port and the second pressure transducer is configured to measure the pressure before the piezoelectric valve.

In some aspects, the techniques described herein relate to a non-invasive blood pressure monitoring system including: a pump system including: a pump including an inlet and an outlet; inlet pressure chambers connected to the inlet, wherein the inlet pressure chambers are connected in series to such that a last inlet pressure chamber is connected to the inlet of the pump and a first inlet pressure chamber is connected to outside air; outlet pressure chambers connected to the outlet, wherein each of the outlet pressure chambers are connected in series such that a first outlet pressure chamber is connected to the outlet of the pump and a last outlet pressure chamber is connected to a port, and a cuff including a bladder connected to the port, wherein the bladder is configured to apply pressure to non-invasively measure blood pressure.

### BRIEF DESCRIPTION OF THE DRAWINGS

The description will be more fully understood with reference to the following figures and data graphs, which are presented as various embodiment of the disclosure and should not be construed as a complete recitation of the scope of the disclosure, wherein:

FIG. 1 provides an example of a mock data graph of a pressure measurement that is typical of a pressure source providing a constant pressure.

FIG. 2 is an overview system diagram of a pressure system utilized within a blood pressure monitoring system.

FIG. 3A is a front view of a portable monitoring system.

FIG. 3B is a top down view of a portable monitoring system.

FIG. 3C is a side view of a portable monitoring system.

FIG. 3D is an enlarged view of an adjustable cuff.

FIG. 3E is an exploded view of an adjustable cuff.

FIG. 4A is a front view of a portable monitoring system.

FIG. 4B is a top down view of a portable monitoring system.

FIG. 4C is a side view of a portable monitoring system.

FIG. 5 is a perspective view of a pump system.

FIG. 6 is an exploded view of a pump system.

FIG. 7 is a perspective view of a pulsation damping manifold.

FIG. 8 is a block diagram of a pulsation damping system in accordance with an implementation.

FIG. 9A is a top perspective view of a pump system.

FIG. 9B is a bottom perspective view of the pump system.

FIG. 10A is the bottom perspective view of the pump system with a pulsation damping manifold visible.

FIG. 10B is a top down view of a portion of the pulsation damping manifold.

FIG. 10C is a top down view of a portion of the pulsation damping manifold.

FIG. 11A is a perspective view of the pump system with the pulsation damping manifold and a piezoelectric valve visible.

FIG. 11B is a perspective view of the pump system with the pulsation damping manifold, the piezoelectric valve, and a PCB visible.

FIG. 12 is a perspective view of the pump system with the pulsation damping manifold, a gasket, and a top plate visible.

FIG. 13A is an enlarged cross-sectional view of the pulsation damping manifold, the gasket, and the top plate.

FIG. 13B is a perspective view of a portion of the pulsation damping manifold.

FIG. 14 is a perspective view of the pump system with the gasket visible.

FIG. 15 is a perspective view of the pump system with the top plate visible.

FIG. 16 is a block diagram of a portable blood pressure monitoring system.

FIG. 17 is a block diagram of a pressure controller.

FIG. 18 is a flow chart of a method of controlling pressure in a blood pressure monitoring system.

### DETAILED DESCRIPTION

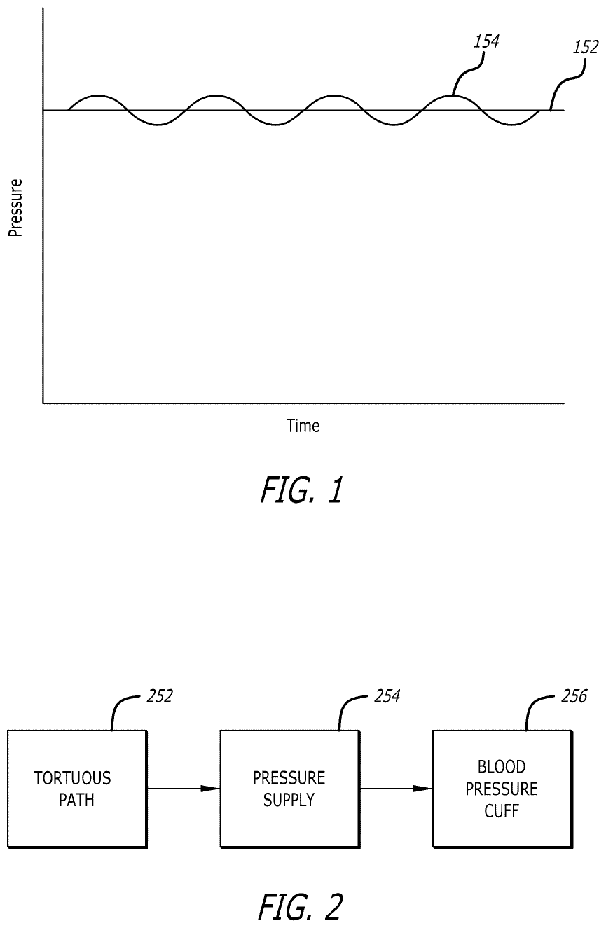

The current disclosure details systems and methods for actively damping pressure in a compact blood pressure monitoring system. Pressure pumps and other pressure sources typically provide pulsating pressure, and although the pulsation is minor, it can cause inaccuracies in sensitive measurements and treatments. FIG. 1 provides an example of a mock data graph of a pressure measurement that is typical of a pressure source providing a constant pressure. While the average pressure 152 provided is constant, the actual provided pressure 154 pulsates up and down, yielding a pulsation effect of the average provided pressure.

One goal of the current application is to provide a compact system for providing continuous blood pressure monitoring. Because a compact system is smaller and would have less chambers, it would typically utilize less pressure to provide continuous blood pressure monitoring. However, with less pressure, there is a greater relative amount of pump pulsations to the average pressure amount provided. Further, as a compact system can be portable, the system may be positioned near the patient and thus patient's comfort would be aided by keeping pump noise minimal. A portable system may be valuable when a dedicated blood monitoring system is not available and/or when a full monitoring system is not necessary. For example, a portable system may be useful in less intensive medical care settings (e.g., not an OR and/or recovery room). Examples of less intensive medical care settings include a doctor's office, field use, and/or at-home professional care.

The disclosed compact systems and devices reduce the pulsation effect that is provided by a pressure source via a tortuous path. The tortuous path can decrease pressure undulations into a more smoothened provided pressure, with less pulsation. The smoothened pressure can yield better accuracy when used in a blood pressure monitoring system. While traditional blood pressure monitoring pump systems may include a damping system, these damping systems are generally large in size with several large chambers (e.g., volume of 17 cc). Pump systems also typically provide a pressure control valve within a separate pressure controller. Such pump systems limit the overall mobility for portable applications due to the size and number of components that they entail.

A compact blood pressure pump system would ideally provide an acceptable level of audible noise when the patient is positioned within the near proximity of the pump system but still provide enough pressure to reduce pump pulsations. A mere reduction of traditional damping systems provided in series separated by restrictive orifices would emit audible noise that would be bothersome to a nearby patient. Thus, non-classical methods of providing and damping pressure are needed to yield an acceptable compact blood pressure monitoring device.

The disclosed pump systems for use in a portable non-invasive blood pressure monitoring system can include a pump, a pressure control valve, and/or a tortuous path. Implementation of a tortuous path can also create unwanted pump noise which is magnified with a longer tortuous path length and thus there is a need to provide a proper balance between reducing pressure pulsations and minimizing audible noise.

Provided in FIG. 2 is an overview system diagram of a pressure system utilized within a blood pressure monitoring system. The pressure system includes a pressure supply 254 and tortuous path 252. Pressure supply 254 can be any source that can provide pressure, such a pressure pump. The pressure pump can be a positive-displacement pump, a centrifugal pump, an axial-flow pump or any other pump capable of generating pressure. In some instances, the pressure pump is a positive-displacement pump. Types of positive-displacement pumps that can be utilized include (but are not limited to) rotary-type pumps, reciprocating-type pumps, linear-type pumps, and pneumatic pumps. The various pumps each provide a relative amount of pressure undulation as the pump mechanics facilitate the movement of fluid (liquid or gas).

The pressure supply 254 provides a pressure by pulling air that passes through the tortuous path 252. The tortuous path 252 reduces the amount of pulsation by requiring greater amount of pressure to be supplied by the tortuous path 252.

Due to the path design, the tortuous path 252 makes it more difficult for the pressure supply 254 to draw air, reducing the pressure pulsations by making the pressure supply work harder. The greater the pressure generated, the less relative effect of the pressure supply 254 to pneumatic pump pulsations. The tortuous path 252 provides a resistance to the pressure, however, creates pump noise pulsations associated with the pressure supply 254. Air can be pulled into an inlet by the pump, and travel through the tortuous path 252 which can be stretched out and can bend repeatedly in a snake-like or zig-zag pattern to be compact. The air exits an outlet as it is pulled into the pump. A filter can also be provided before, after, or integrated within the tortuous path 252.

To reduce pressure pulsations and keep audible noise minimal, it has been found that a tortuous path should have a length to diameter ratio of about 150:1 to 200:1. In other words, the length of the tortuous path should be 150-times to 200-times longer than its diameter. In various implementations, the tortuous path is: about 150-times longer than its diameter, about 160-times longer than its diameter, about 170-times longer than its diameter, about 180-times longer than its diameter, about 190-times longer than its diameter, or about 200-times longer than its diameter. In some implementations, the tortuous path is between 18 inches in length and 30 inches in length. In various implementations, the tortuous path is: about 18 inches in length, about 20 inches in length, about 22 inches in length, about 24 inches in length, about 26 inches in length, about 28 inches in length, about 30 inches in length, about 32 inches in length, about 34 inches in length, or about 36 inches in length.

When the pressure supply 254 is utilized within blood pressure monitoring system, the pressure outflow out of the pressure supply 254 is utilized for a cuff 256 to perform blood pressure readings on an extremity. In some implementations, a damping system may be provided in the path between the pressure supply 254 and the cuff 256. The damping system may include an active damping system and/or one or more passive damping systems. In some implementations that utilize an active and a passive damping system, the one or more passive damping systems can be provided before, after, or integrated within the active damping system. The passive damping systems may include expansion chambers and/or alter the direction of pressure flow to damp pressure pulsation. Passive damping systems or active damping systems may be utilized to fit a certain compact size blood pressure monitoring system. Traditional passive damping systems may not be compact however active damping can be utilized to decrease the size of the blood pressure monitoring system. The active damping system may include a poppet system which may decrease the size of the blood pressure monitoring system.

The damped pressure outflow may travel through a pressure control system that senses the pressure amount and can adjust the supplied pressure. The pressure is then transferred into the blood pressure cuff 256, which can be a cuff surrounding any extremity of a patient, such as (for example) an arm or a digit. In some instances, the blood pressure monitoring system utilizes a volume clamp method for continuous blood pressure monitoring and thus the pressure is adjusted based on the amount of pressure to keep the diameter of a patient's artery constant via the blood pressure cuff 256. When a volume clamp method is utilized, the supplied pressure to the cuff that keeps the artery diameter constant is the blood pressure within that artery.

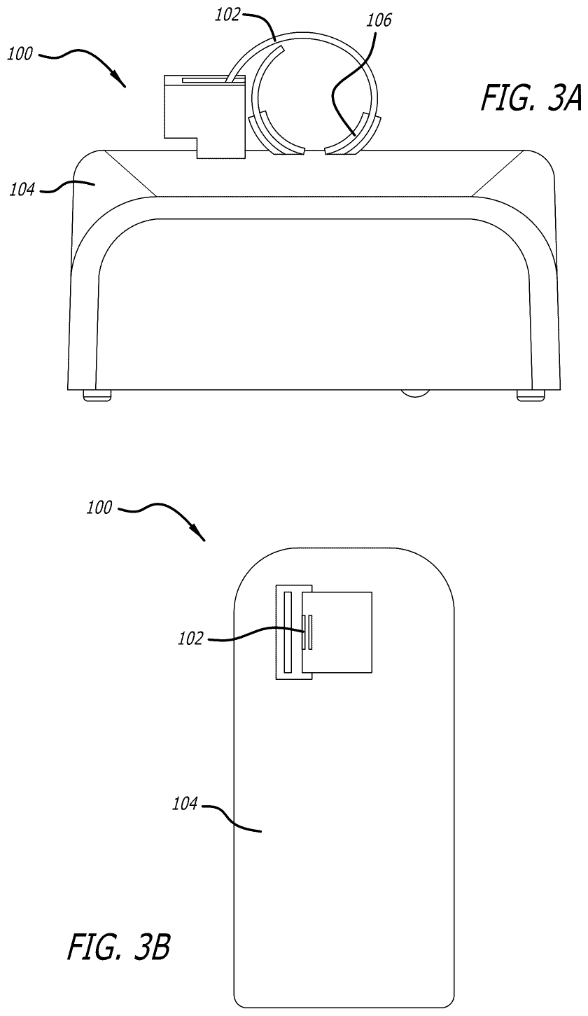

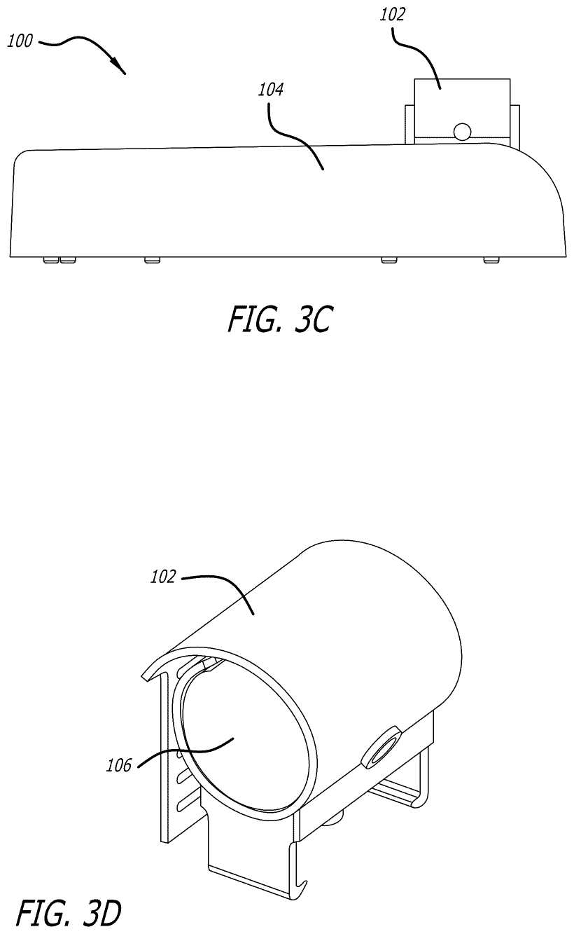

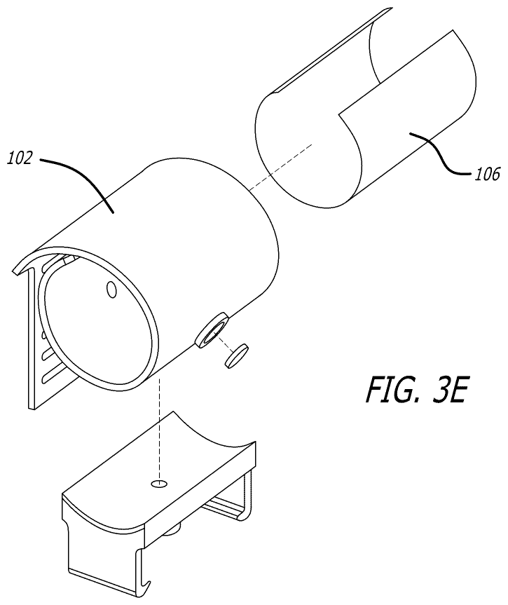

FIGS. 3A-3E illustrate various views of the portable monitoring system. As illustrated, the portable monitoring system 100 includes an adjustable cuff 102 which is connected to a pump system 104. The adjustable cuff 102 incorporates a bladder 106. FIG. 3D is an enlarged view of the adjustable cuff 102. FIG. 3E is an exploded view of the adjustable cuff 102. As illustrated, the bladder 106 is incorporated within the cuff 102.

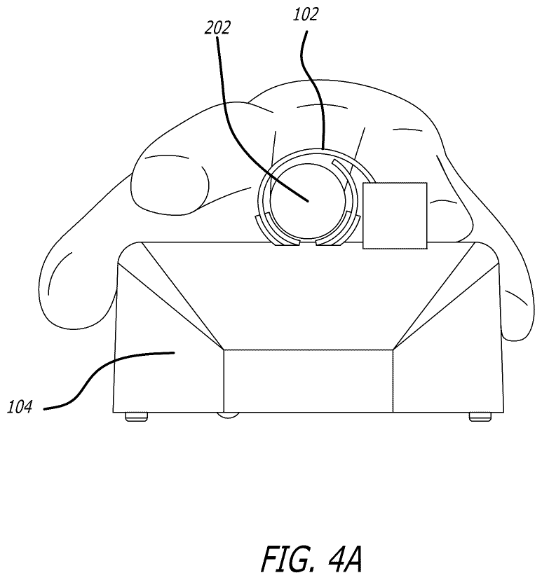

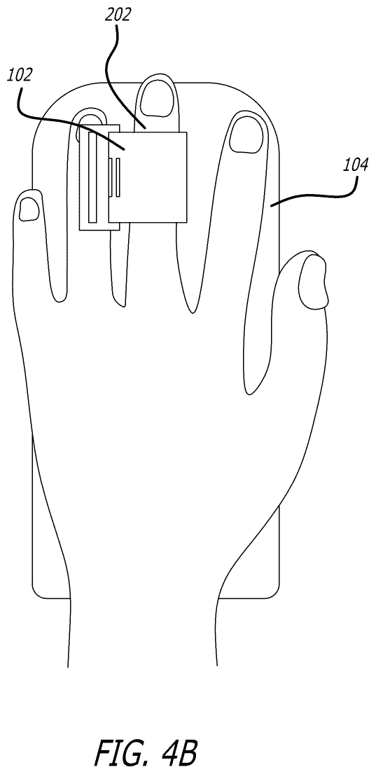

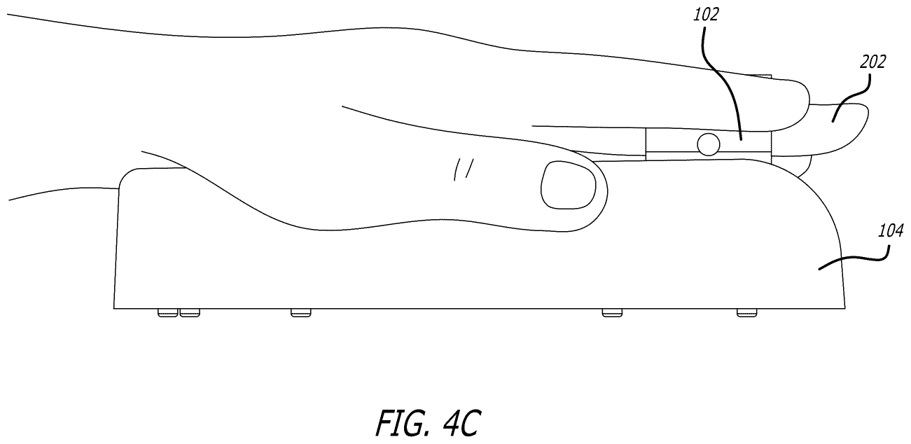

FIGS. 4A-4C illustrate various views of the portable monitoring system described in connection with FIGS. 3A-3C while being on a patient's hand. FIG. 4A is a front view of the portable monitoring system. FIG. 4B is a top down view of the portable monitoring system. FIG. 4C is a side view of the portable monitoring system. As illustrated, the patient's finger 202 goes into the adjustable cuff 102. The cuff 102 includes the bladder 106 which constricts the patient's finger which provides a mechanism for sensing the patient's blood pressure. The bladder 106 utilizes the pump system 104.

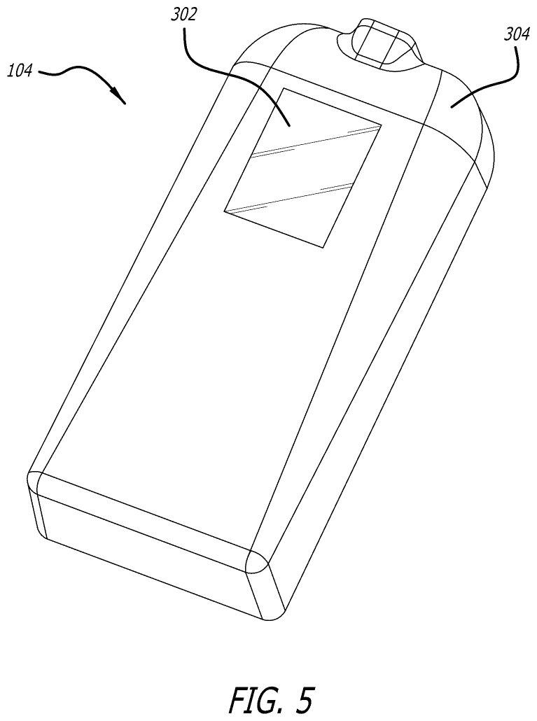

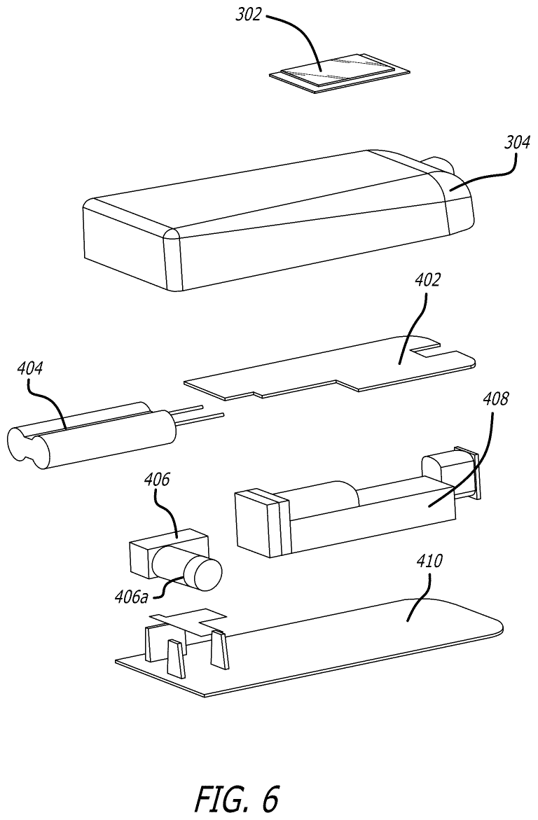

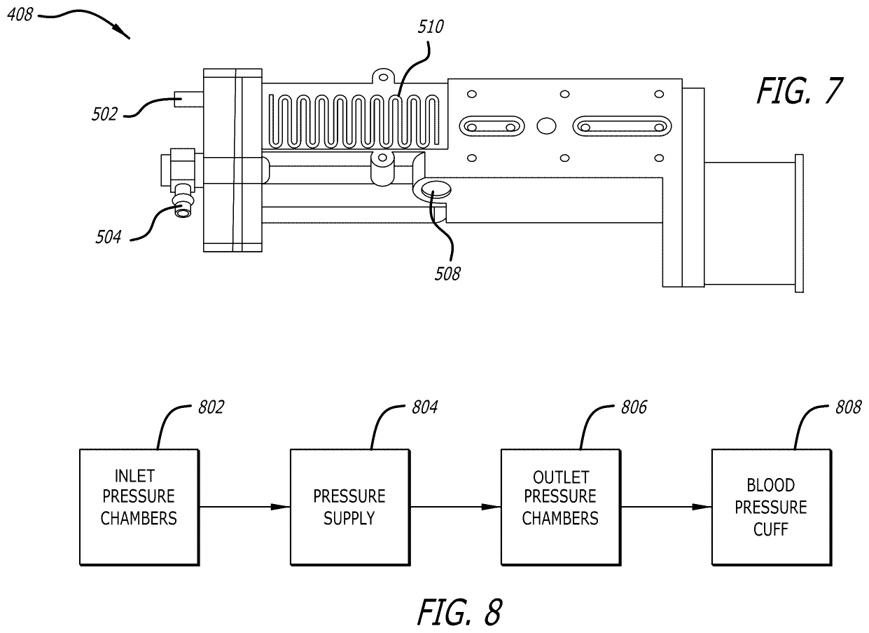

FIG. 5 is a perspective view of the pump system 104. As illustrated, the pump system 104 includes a display 302 (e.g. an LCD) which is cased in a housing 304. FIG. 6 is an exploded view of the pump system 104. The pump system 104 includes a printed circuit board assembly (PCBA) 402 including a printed circuit board. The printed circuit board assembly 402 is powered by a battery 404 and is used to control a pump 406 (e.g. a pneumatic pump). The pump 406 is connected to a pulsation damping manifold 408. A more detailed view of the pulsation damping manifold 408 is described in connection with FIG. 7. The housing 304 and a baseplate 410 cooperate to house PCBA 402, battery 404, the pump 406 and the pulsation damping manifold 408. The pump 406 may be fitted with or attached to an orifice 406a. The pump 406 may be attached to one or more volume accumulators which may receive pressure from the pump 406. The volume of the volume accumulator may be specific to the pump 406. For example, the volume of the volume accumulator may be related to the stroke volume of the pump 406. The stroke volume of pump 406 is how far the diaphragm of the pump 406 moves back and forth. The stroke volume of the pump 406 may be equal to the volume of the volume accumulator. The orifice 406a may have a diameter of 0.1 times the stroke volume of the pump 406 and/or the volume of the volume accumulator. The smaller the orifice diameter, the more the pump 406 works and thus the orifice diameter is balanced with the pump 406 power. The pump 406 is connected to the bladder 106.

In some examples, there may be one or more active damping units (e.g. poppets or bellows) used in place of the orifice 406a. Examples of active damping units are described in Int. App. No. PCT/US2023/076265, entitled “Systems and Methods for Responsive Damping of Pressure” and filed Oct. 6, 2023 which is hereby incorporated by reference in its entirety for all purposes. The one or more active damping units can measure pressure or volume ripples and can control flow coming in. The one or more active damping units may include a bellow which expands and has an orifice 406a which bleeds out. Control of the one or more active damping units may be based on a spring as volume comes in, fills in and expands which would require less power and be quieter.

FIG. 7 is a perspective view of the pulsation damping manifold 408. The pulsation damping manifold 408 includes an inlet 504. Fresh air is sucked through the inlet 504 and through an inlet filter 508. The inlet filter 508 may filter 40 microns size particles. The air passes from the inlet filter 508 through a tortuous path 510. As discussed previously, the tortuous path reduces pump pulsations but also creates pump noise. The tortuous path 510 outputs air into an output 502 which is connected to the pump 406. The inlet 504 of the tortuous path 510 is configured to receive outside air such that the received air travels through the tortuous path 510 prior to entering the pump 406. The pump 406 provides pressure to the inlet 504 which is fed through an inlet filter 508. The pump 406 may run at 50 hertz. The pump 406 may be connected to a bladder 106 which applies pressure to the patient's finger.

To reduce pressure pulsations and keep audible noise minimal, it has been found that a tortuous path should have a length to diameter ratio of about 150:1 to 200:1. In other words, the length of the tortuous path should be 150-times to 200-times longer than its diameter. In various implementations, the tortuous path is: about 150-times longer than its diameter, about 160-times longer than its diameter, about 170-times longer than its diameter, about 180-times longer than its diameter, about 190-times longer than its diameter, or about 200-times longer than its diameter. In some implementations, the tortuous path is between 18 inches in length and 30 inches in length. In various implementations, the tortuous path is: about 18 inches in length, about 20 inches in length, about 22 inches in length, about 24 inches in length, about 26 inches in length, about 28 inches in length, about 30 inches in length, about 32 inches in length, about 34 inches in length, or about 36 inches in length. The yielded volume may provide an advantageous pressure to noise ratio. While the tortuous path 510 is illustrated to bend and snake, this bending merely makes the tortuous path 510 more compact and thus other shapes that yield similar lengths may yield similar results. The number of bends and the slope of the bends may be different than the example illustrated. The tortuous path 510 may be elongated and snake back and forth laterally. The tortuous path 510 may have a circular cross-section with a diameter and a path length. The tortuous path 510 may be passive with no moving elements.

It some examples, the pulsation damping manifold may be implemented utilizing pressure chambers. The pressure chambers may be utilized at the inlet of the pressure supply and the outlet of the pressure supply. FIG. 8 is a block diagram of a pulsation damping system in accordance with an implementation. The pulsation damping system may include inlet pressure chambers 802 which may be connected to the inlet of a pressure supply 804. The pressure supply 804 may output pressure to outlet pressure chambers 806. The outlet pressure chambers 806 are in connection with one or more blood pressure cuffs 808. The pressure may be transferred from the pressure supply 804 to the outlet pressure chambers 806 into the blood pressure cuffs 808. The blood pressure cuffs 808. The blood pressure cuffs 808 may surround any extremity of a patient, such as (for example) an arm or a digit. In some instances, the blood pressure monitoring system utilizes a volume clamp method for continuous blood pressure monitoring and thus the pressure is adjusted based on the amount of pressure to keep the diameter of a patient's artery constant via the blood pressure cuffs 808. When a volume clamp method is utilized, the supplied pressure to the cuff that keeps the artery diameter constant is the blood pressure within that artery.

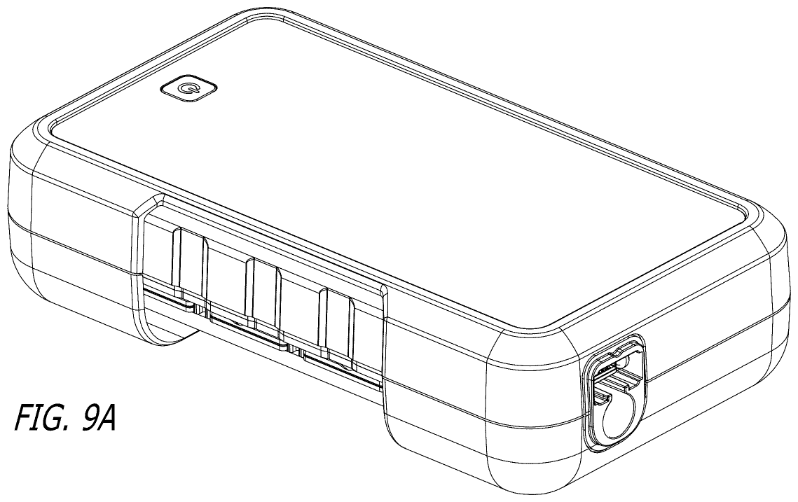

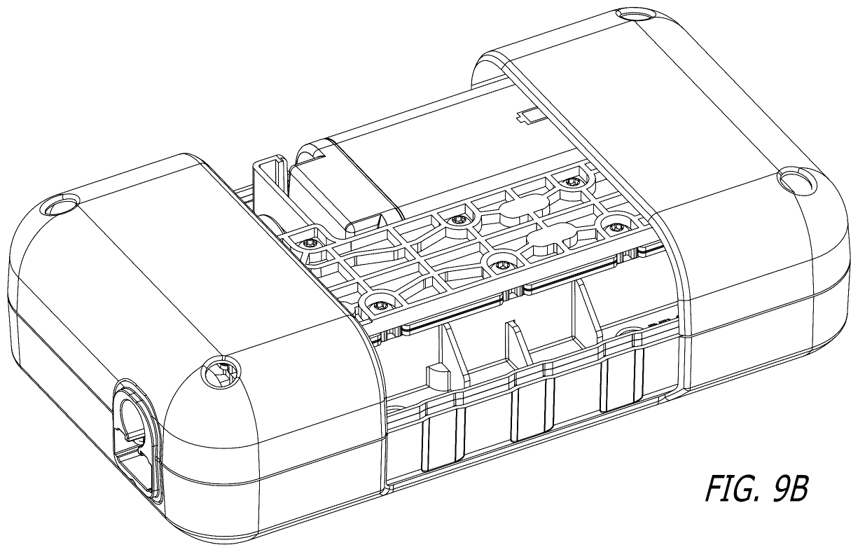

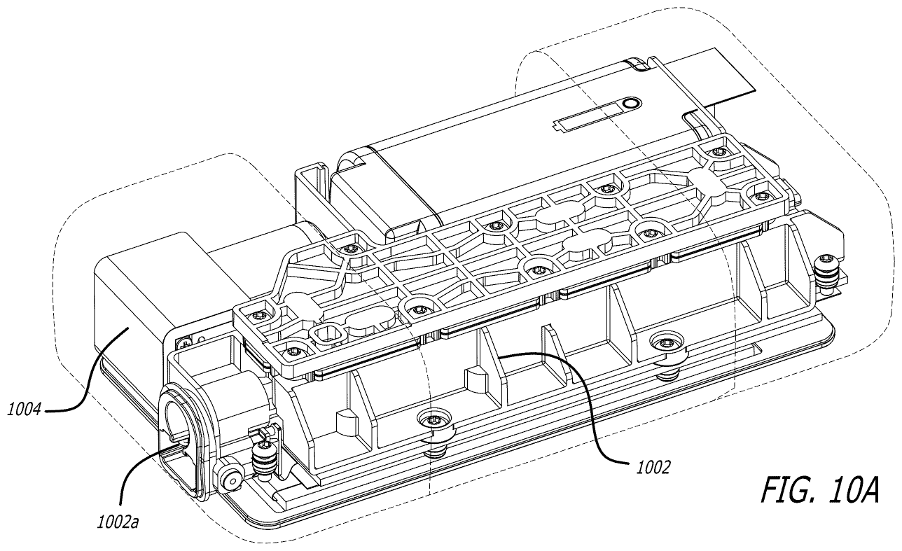

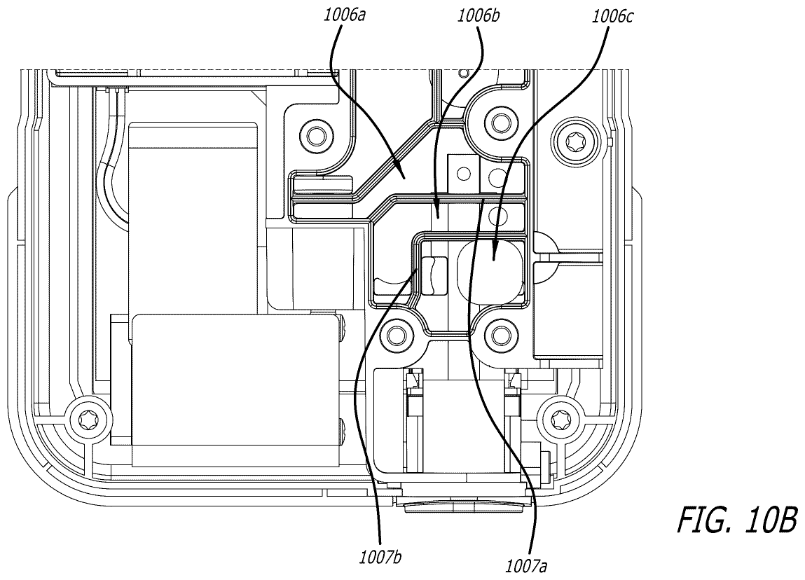

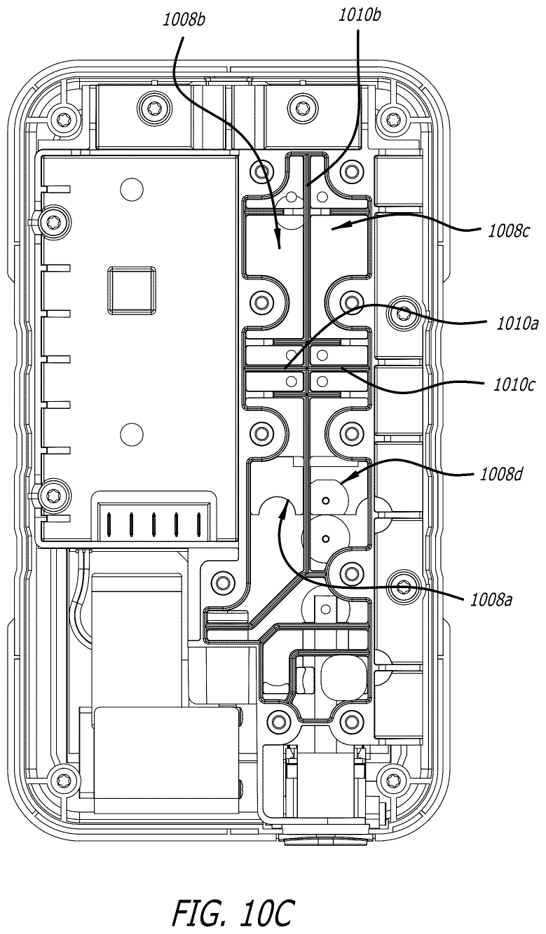

The inlet pressure chambers 802 and the outlet pressure chambers 806 may be implemented in one or more pulsation damping manifolds. FIG. 9A is a top perspective view of a pump system. FIG. 9B is a bottom perspective view of the pump system. FIG. 10A is the bottom perspective view of the pump system with a pulsation damping manifold 1002 visible. The pulsation damping manifold 1002 is in pressure connection with a pump 1004. The pulsation damping manifold 1002 includes a port 1002a which may be connected to one or more blood pressure cuffs. FIG. 10B is a top down view of a portion of the pulsation damping manifold 1002. The pulsation damping manifold 1002 includes a first inlet pressure chamber 1006a, a second inlet pressure chamber 1006b, and a third inlet pressure chamber 1006c. The first inlet pressure chamber 1006a is divided from the second inlet pressure chamber 1006b by a first inlet partition 1007a. The second inlet pressure chamber 1006b is divided from the third inlet pressure chamber 1006c by a second inlet partition 1007b. A first inlet passage connects the first inlet pressure chamber 1006a and the second inlet pressure chamber 1006b above the first inlet partition 1007a. A second inlet passage connects the second inlet pressure chamber 1006b and the third inlet pressure chamber 1006c above the second inlet partition 1007b. The first inlet passage and the second inlet passage are described in detail below. The first inlet pressure chamber 1006a is connected to the inlet of the pump 1004. The pump 1004 pulls air through the inlet and thus through the first inlet pressure chamber 1006a, the second inlet pressure chamber 1006b, and the third inlet pressure chamber 1006c. The first inlet pressure chamber 1006a is the last inlet pressure chamber which is connected to the inlet of the pump 1004. The third inlet pressure chamber 1006c is the beginning inlet pressure chamber which is connected to outside air.

FIG. 10C is a top down view of a portion of the pulsation damping manifold 1002. The pulsation damping manifold 1002 includes a first outlet pressure chamber 1008a, a second outlet pressure chamber 1008b, a third outlet pressure chamber 1008c, and a fourth outlet pressure chamber 1008d. The first outlet pressure chamber 1008a is divided from the second outlet pressure chamber 1008b by a first outlet partition 1010a. The second outlet pressure chamber 1008b is divided from the third outlet pressure chamber 1008c by a second outlet partition 1010b. The third outlet pressure chamber 1008c is divided from the fourth outlet pressure chamber 1008d by a third outlet partition 1010c. A first outlet passage connects the first outlet pressure chamber 1008a and the second outlet pressure chamber 1008b above the first outlet partition 1010a. A second outlet passage connects the second outlet pressure chamber 1008b and the third outlet pressure chamber 1008c above the second outlet partition 1010b. A third outlet passage connects the third outlet pressure chamber 1008c and the fourth outlet pressure chamber 1008d above the third outlet partition 1010c. The first outlet passage, the second outlet passage, and the third outlet passage are described in detail below. The fourth outlet pressure chamber 1008d is connected to the port 1002a which may be connected to one or more blood pressure cuffs. The first outlet pressure chamber 1008a is connected to the outlet of the pump 1004.

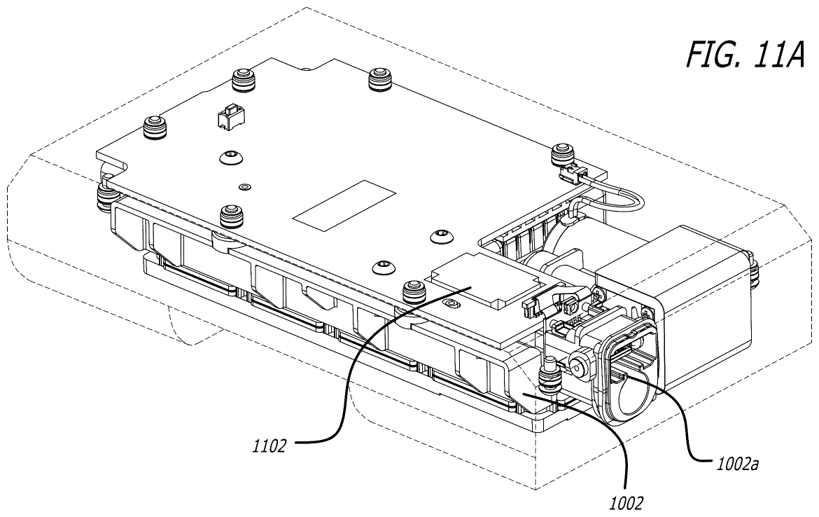

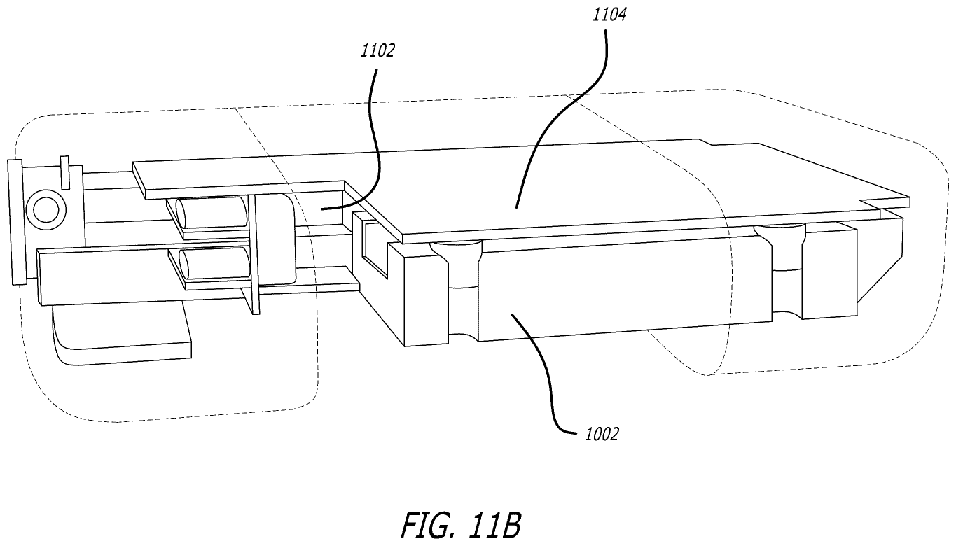

FIG. 11A is a perspective view of the pump system with the pulsation damping manifold 1002 and a piezoelectric valve 1102 visible. The fourth outlet pressure chamber 1008d may be connected to the port 1002a through the piezoelectric valve 1102. The piezoelectric valve 1102 may have a first setting and a second setting. The first setting connects the fourth outlet pressure chamber 1008d to the port 1002a. The second setting connects the fourth outlet pressure chamber 1008d and the port 1002a to a vent. The vent may be utilized to bring the first outlet pressure chamber 1008a, the second outlet pressure chamber 1008b, the third outlet pressure chamber 1008c, and the fourth outlet pressure chamber 1008d and the one or more blood pressure cuffs connected through the port 1002a to room pressure while the pump 1004 is not in operation. Advantageously, this single piezoelectric valve 1102 easily vents the one or more blood pressure cuffs and also reduces pressure in the outlet pressure chambers.

The pump system may include two pressure transducers. FIG. 11B is a perspective view of the pump system with the pulsation damping manifold 1002, the piezoelectric valve 1102, and a PCB 1104 visible. The PCB 1104 may include two pressure transducers which are located adjacent to the piezoelectric valve 1102. A first pressure transducer may be located adjacent to the piezoelectric valve 1102 to sense the pressure going into the cuff through the port 1002a. In other pump systems, this pressure transducer was located through a tube off of the port 1002a. The pump system utilizes pressure measurements from the first pressure transducer to regulate the pressure within the cuff and adjust the operation of the pump 1004. A second pressure transducer may also be located adjacent to the piezoelectric valve and may sense the pressure before the piezoelectric valve 1102. The first pressure transducer measures cuff pressure and is used to control the piezo valve which controls the pressure in the cuff. The second pressure transducer measures the manifold pressure and is used to control the pump to maintain constant pressure in manifold. Each of these pressure transducers are located on a printed circuit board (PCB) which is located in the casing of the pump system. In previous pump systems, the pump system is separated from the port (analogous to the port 1002a of FIG. 11B) and is connected to the port via a flexible tubing. In previous pump systems, the pressure sensor (e.g. transducer) used to measure cuff pressure is connected via pathways in the port geometry.

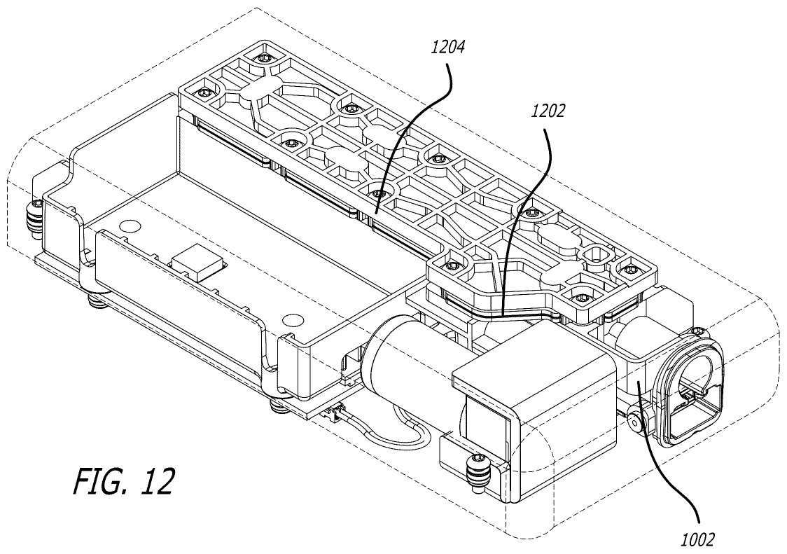

Each of the passages includes the first inlet passage, the second inlet passage, the first outlet passage, the second outlet passage, the third outlet passage, and the fourth outlet passage may be formed utilizing a gasket positioned between a top plate and the pulsation damping manifold 1002. FIG. 12 is a perspective view of the pump system with the pulsation damping manifold 1002, a gasket 1202 and a top plate 1204 visible. The gasket 1202 may be positioned between the pulsation damping manifold 1002 and the top plate 1204 such that air travels from one chamber through the gasket to the top plate 1204 and back through the gasket and back into another chamber.

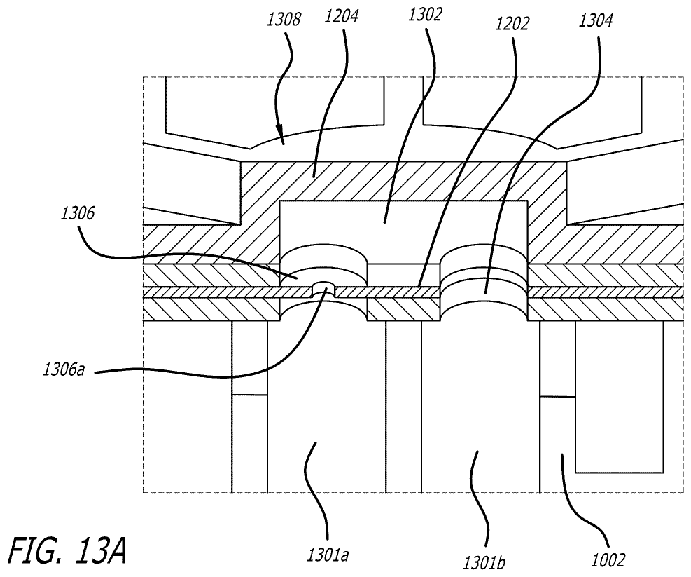

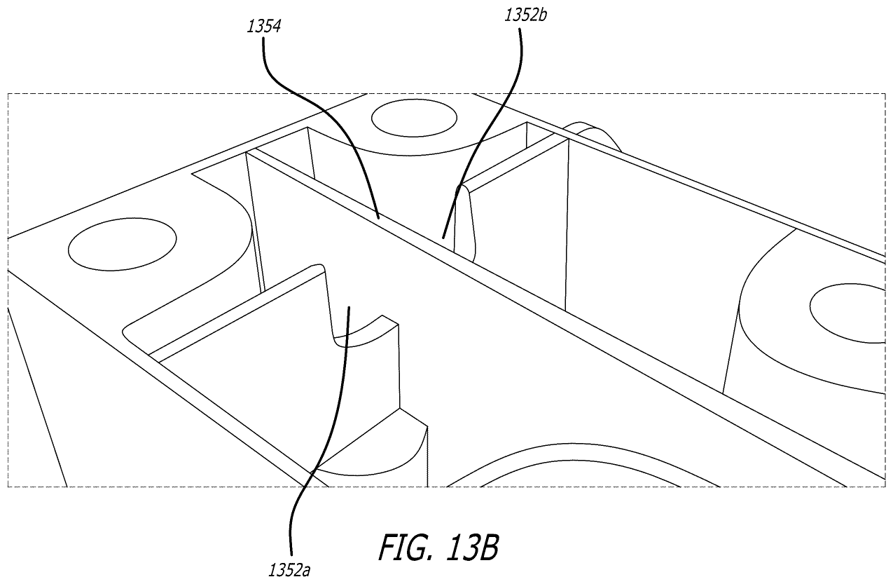

FIG. 13A is an enlarged cross-sectional view of the pulsation damping manifold 1002, the gasket 1202, and the top plate 1204. The pulsation damping manifold 1002 includes a first chamber 1301a and a second chamber 1301b. The first chamber 1301a and the second chamber 1301b are adjacent and connected through a passage 1308. The passage 1308 is formed through the gasket 1202 and the top plate 1204. The gasket 1202 is positioned between the pulsation damping manifold 1002 and the top plate 1204 and creates a seal between the pulsation damping manifold 1002 and the top plate 1204. The gasket 1202 includes a first opening 1306 and a second opening 1304. The first opening 1306 includes an orifice 1306 which is a thin disk shaped member including a small hole 1306a. The hole 1306a is smaller than the first opening 1306. The orifice 1306 is thinner than the gasket. In some examples, the gasket 1202 may include two thick flexible sheets and a thin flexible sheet positioned between the two thick flexible sheets. The thin flexible sheet may include the small hole 1306a. In some examples the orifice may be a thin flexible sheet which only resides among the immediate vicinity of the first opening 1306. The orifice 1306 may alleviate much of the noise which is created during the operation of the pump. The first opening 1306 and the second opening 1304 are connected by a channel 1302 within the top plate 1204. Air passes from the first chamber 1301a through the hole 1306a within the orifice 1306 through the channel and through the second opening 1304 and into the second chamber 1301b. In some examples, the orifice 1306 may be present in the second opening 1304 and not in the first opening 1306.

In the first inlet passage 1007a and the second inlet passage 1007b, the orifice 1306 is positioned such that air is pulled through the first chamber 1301a, through the orifice 1306, through the channel 1302, through the second opening 1304, and into the second chamber 1301b. In some implementations, the position of the orifice 1306 and the second opening 1304 may be swapped such that air is pulled through the first chamber 1301a, through the second opening 1304, through the channel 1302, through the orifice 1306, and into the second chamber 1301b. In some implementations, there an orifice may also be in the second opening 1304 such that air is pulled through the first chamber 1301a, through the orifice 1306, through the channel 1302, through the orifice in the second opening 1304 and into the second chamber 1301b.

In the first outlet passage 1010a, the second outlet passage 1010b, and the third outlet passage 1010c, the orifice 1306 is positioned such that air is pushed through the first chamber 1301a, through the orifice 1306, through the channel 1302, through the second opening 1304, and into the second chamber 1301b. In some implementations, the position of the orifice 1306 and the second opening 1304 may be swapped such that air is pulled through the first chamber 1301a, through the second opening 1304, through the channel 1302, through the orifice 1306, and into the second chamber 1301b. In some implementations, there an orifice may also be in the second opening 1304 such that air is pulled through the first chamber 1301a, through the orifice 1306, through the channel 1302, through the orifice in the second opening 1304 and into the second chamber 1301b.

FIG. 13B is a perspective view of a portion of the pulsation damping manifold 1002. As described above, the pulsation damping manifold 1002 includes partitions 1354 that separate the various inlet and outlet pressure chambers. Adjacent to each of the partitions 1354, may be a first cutout 1352a which corresponds to one of the pressure chambers and a second cutout 1352b which corresponds to another of the pressure chambers. The gasket 1202 presses against the partitions 1354 and the first cutout 1352a and the second cutout 1352b allow the gasket 1202 to flex to create a good seal against the partitions 1354.

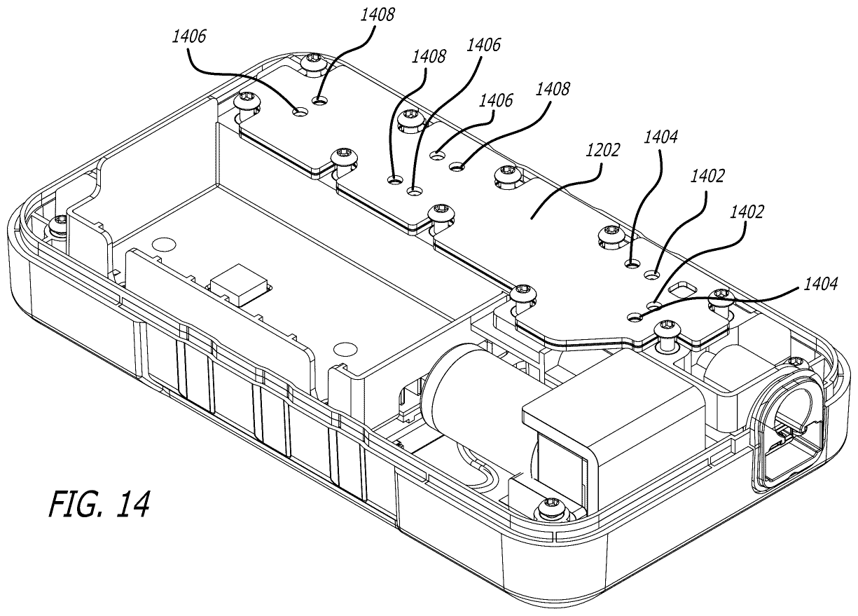

FIG. 14 is a perspective view of the pump system with the gasket 1202 visible. As previously described, the pulsation damping manifold 1002 includes pressure chambers corresponding to the inlet of the pump and pressure chambers corresponding to the outlet of the pump. Similarly, the gasket 1202 includes openings which correspond to the inlet of the pump and openings corresponding to the outlet of the pump.

For the openings corresponding to the inlet, air is pulled from a chamber of the pulsation damping manifold 1002 through an orifice 1402, through an opening 1404 without an orifice and into another chamber of the pulsation damping manifold 1002. This is repeated until the air is pulled through the pump.

For the openings corresponding to the outlet, air is pushed from a chamber of the pulsation damping manifold 1002 through an orifice 1406, through an opening 1408 without an orifice and into another chamber of the pulsation damping manifold 1002. This is repeated until the air is pushed to the port which may be connected to one or more blood pressure cuffs.

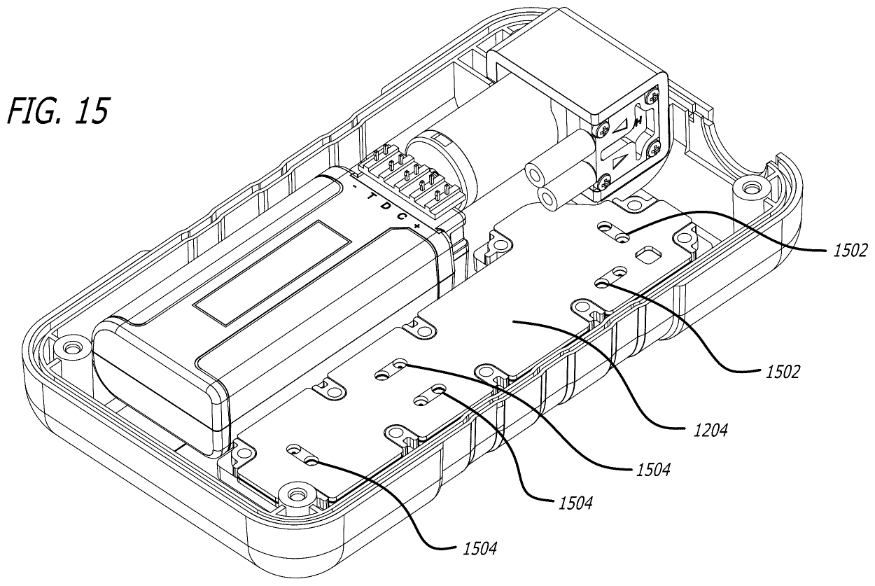

FIG. 15 is a perspective view of the pump system with the top plate 1204 visible. As previously described, the pulsation damping manifold 1002 includes pressure chambers corresponding to the inlet of the pump and pressure chambers corresponding to the outlet of the pump. Similarly, the top plate 1204 includes channels 1502 which correspond to the inlet of the pump and channels 1504 which correspond to the outlet of the pump.

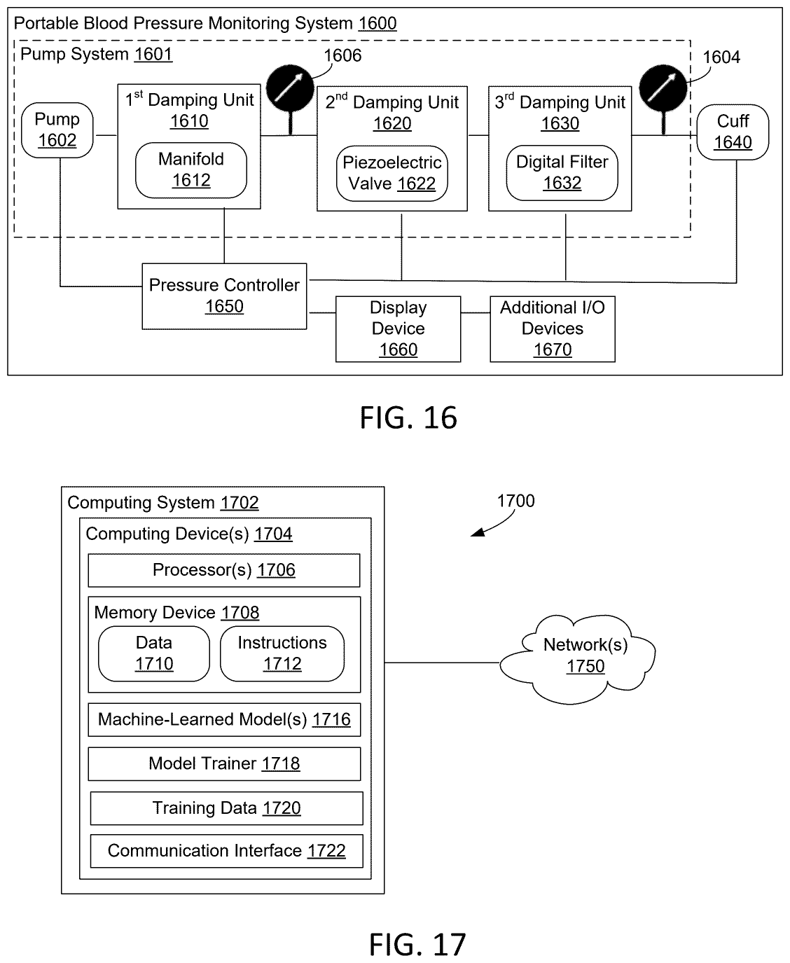

FIG. 16 is a block diagram of a portable blood pressure monitoring system according to example embodiments of the present disclosure. The portable blood pressure monitoring system 1600 generally provides air pressure from a pump system 1601 to a cuff 1640. The pump system 1601 can include integrated pressure control features such as one or more damping units configured to damp the air pressure and one or more transducers configured to measure the air pressure. By integrating various pressure control features directly within the pump system 1601, overall function of the portable blood pressure monitoring system can be improved by establishing a more constant pressure source with reduced pulsations or ripples in fluid volume.

In the example embodiment of FIG. 16, the pump system 1601 of portable blood pressure monitoring system 1600 can include a pump 1602, a first pressure transducer 1604, a second pressure transducer 1606, a first damping unit 1610, a second damping unit 1620, a third damping unit 1630, a pressure controller 1650, a display device 1660, and additional input/output (I/O) devices 1670.

The portable blood pressure monitoring system 1600 can include a housing configured to encase the pump system 1601 including the pump 1602, the first damping unit 1610, the second damping unit 1620 and the third damping unit 1630, as well as pressure controller 1650. The cuff 1640 can be positioned on a surface of the housing, such as depicted in FIGS. 4A-4C.

The pump 1602 generally can be configured to provide a pressure source. In some examples, the pump 1602 of FIG. 16 can correspond to the pressure supply 254 of FIG. 2 or any source that can provide pressure, such a pressure pump. The pump 1602 can include an inlet and an outlet respectively coupled to other components of the portable blood pressure monitoring system 1600, including but not limited to the first damping unit 1610, the pressure controller 1650, etc.

The first damping unit 1610 can be coupled to the pump 1602 such that a pressure source output of the pump 1602 is received by the first damping unit 1610. The first damping unit 1610 can be generally configured to pneumatically damp a pressure pulsation of the pressure source provided by the pump 1602 by a first amount of damping. In other words, volume ripples within the pressure signal provided by the pump 1602 can be reduced by the first amount of damping. The first damping unit 1610 can include a manifold 1612 configured to implement the pneumatic damping by constricted channeling of fluid using one or more mechanisms disclosed herein (e.g., multiple pressure chambers, specially configured partitions among connected pressure chambers, tortuous path(s) for fluid flow, etc.)

The manifold 1612 can correspond to any of the pulsation damping manifolds disclosed herein, including but not limited to the pulsation damping manifold 408 of FIGS. 6-7 and/or the pulsation damping manifold 1002 of FIGS. 10A-10C, 11A-11B, 12, and 13A-13B. The manifold 1612 can include one or more pressure chambers connected in series. In some examples, the manifold 1612 can include a first pressure chamber and a second pressure chamber. The first pressure chamber and the second pressure chamber of the manifold 1612 can be separated by a partition. Example partitions are variously depicted in and described with reference to FIGS. 10B-10C and 13A-13B. The first pressure chamber and the second pressure chamber of the manifold 1612 can additionally or alternatively be connected in series through an arrangement that pneumatically damps pressure between chambers by forcing fluid through a first opening, a channel, and a second opening provided, for example, at a partition between pressure chambers.

In one example, the manifold 1612 can include one or more inlet pressure chambers connected to an inlet of the pump 1602. The inlet pressure chambers of the manifold 1612 can be connected in series to such that a last inlet pressure chamber is connected to the inlet of the pump 1602 and a first inlet pressure chamber is connected to outside air. In some examples when manifold 1612 includes one or more inlet pressure chambers, the inlet pressure chambers can correspond to the inlet pressure chambers 802 of FIG. 8 or the inlet pressure chambers 1006a, 1006b, 1006c of FIG. 10B.

In another example, the manifold 1612 can include one or more outlet pressure chambers connected to an outlet of the pump 1602. The outlet pressure chambers of the manifold 1612 can be connected in series such that a first outlet pressure chamber is connected to the outlet of the pump 1602 and a last outlet pressure chamber is connected to a port of the manifold 1612 (e.g., the port 1002a of FIG. 10A) or housing encasing the portable blood pressure monitoring system 1600. In some examples when manifold 1612 includes one or more outlet pressure chambers, the outlet pressure chambers can correspond to the outlet pressure chambers 806 of FIG. 8 or the outlet pressure chambers 1008a, 1008b, 1008c, 1008d of FIG. 10C.

In another example, the manifold 1612 can include a tortuous path that constricts the flow of fluid contributing to the pressure source within pump system 1601. When manifold 1612 includes a tortuous path, it can correspond to tortuous path 252 of FIG. 2 or tortuous path 510 of FIG. 7. For example, the tortuous path of the manifold 1612 can include a plurality of bends through which fluid (e.g., air) forming the pressure source of pump system 1601 passes through prior to entering or after exiting the pump 1602.

Referring still to FIG. 16, the second damping unit 1620 can be coupled to an output of the first damping unit 1610 and can be generally configured to electrically damp a pressure pulsation of the pressure source provided by the pump 1602 by a second amount of damping. In other words, volume ripples within the pressure signal provided by the pump 1602 can be further reduced by the second amount of damping. The second damping unit 1620 can include a valve 1622 configured to implement electric damping by receiving electric signals indicative of pressure within the pump system 1601 and controlling a position or state of the valve 1622 to increase or decrease fluid flow and corresponding pressure within the pump system 1601.

The valve 1622 can be a piezoelectric valve, such as but not limited to piezoelectric valve 1102 depicted in and described with reference to FIGS. 11A-11B. For example, valve 1622 can be switchable between one or more settings, such as a first setting in which one or more pressure chambers of the manifold 1612 are connected to a port, and a second setting in which both the one or more pressure chambers of the manifold 1612 and the port are connected to outside air.

The valve 1622 of second damping unit 1620 can be coupled to one or more of the first pressure transducer 1604 or second pressure transducer 1606. For example, the valve 1622 can be positioned in between the first pressure transducer 1604 and the second pressure transducer 1606. The first pressure transducer 1604 can be configured to measure pressure at the output port of (after) the pump system 1601 or at the input of (before) the cuff 1640. The first pressure transducer 1604 can thus be configured to determine or generate an output signal indicative of a cuff pressure entering the bladder of the cuff 1640. The second pressure transducer 1606 can be configured to measure pressure at the output of (after) the manifold 1612 or at the input of (before) the valve 1622. The second pressure transducer 1606 can thus be configured to determine or generate an output signal indicative of a manifold pressure exiting the manifold 1612 of first damping unit 1610.

The valve 1622 can be controlled based on pressure signals measured by the first pressure transducer 1604 and/or the second pressure transducer 1606. For example, a first pressure signal measured by first pressure transducer 1604 can be relayed either directly to valve 1622 or through pressure controller 1650 to valve 1622 in order to adjust a state or position of valve 1622 to vent pressure at the cuff 1640. Similarly, a second pressure signal measured by second pressure transducer 1606 can be relayed either directly to valve 1622 or through pressure controller 1650 to valve 1622 in order to adjust a state or position of valve 1622 to vent pressure at the manifold 1612. When the first pressure signal measured and output by the first pressure transducer 1604 and the second pressure signal measured and output by the second pressure transducer 1606 are provided to the pressure controller 1650, the pressure controller 1650 can be further configured to generate a pump control signal for adjusting one or more parameters (e.g., volume, flow rate, pressure level, pressure pulsations, etc.) of the pressure source provided by the pump 1602 and/or generate a cuff control signal for adjusting one or more parameters (e.g., volume, flow rate, pressure level, pressure pulsations, etc.) of the supplied pressure provided by the bladder of the cuff 1640.

Referring still to FIG. 16, the third damping unit 1630 can be coupled to an output of the second damping unit 1620 and can be generally configured to digitally damp a pressure pulsation of the pressure source provided by the pump 1602 by a third amount of damping. In other words, volume ripples within the pressure signal provided by the pump 1602 can be further reduced by the third amount of damping. The third damping unit 1630 can include a filter (e.g., a digital filter) 1632 configured to implement digital damping by employing a software filter to further reduce any volume ripples still remaining in the pressure signal damped by the manifold 1612 and the piezoelectric valve 1622. Filter 1632 can thus be configured to generate a filtered pressure signal characterized by further reduction in pressure pulsation compared with reductions obtained by the first damping unit 1610 and/or second damping unit alone. For example, filter 1632 can be configured to: receive a first signal indicate of supplied pressure (e.g., from the second damping unit 1620) as an input, generate a second signal that filters volume ripples in the first signal, and provide the second signal as an output (e.g., from the third damping unit 1630). The filter output from third damping unit 1630 can be provided either to the cuff 1640 to adjust the supplied pressure provided to the bladder of the cuff 1640 and/or to the pump 1602 to control one or more parameters of the pressure source provided by the pump 1602. In some examples, an output of third damping unit 1630 is provided first to pressure controller 1650 before relay to the pump 1602 and/or cuff 1640.

The digital filter 1632 can be implemented in software, such as within the processor(s) and memory computing functionality of pressure controller 1650 (described with further reference to FIG. 17). Digital filter 1632 can be a finite impulse response (FIR) filter, which is a nonrecursive filter configured to generate a filtered output signal that is computed as a weighted moving average of a given input by using the current and previous inputs to the filter. For example, an FIR filter may use a limited number of coefficients to produce a response to an impulse that has a finite duration. Digital filter 1632 can also correspond to other particular filters, such as but not limited to non-recursive filters, convolution filters, or moving-average filters.

After a pressure source provided by pump 1602 is adjusted according to the first amount of damping obtained by the first damping unit 1610, the second amount of damping obtained by the second damping unit 1620 and the third amount of damping obtained by the third damping unit 1630, the pump system 1601 can provide a beneficially adjusted supplied pressure to the cuff 1640. Cuff 1640 can include a bladder configured to provide the supplied pressure adjusted according to the various amounts of damping to an extremity (e.g., a finger) of a patient. The blood pressure of the patient can then be determined as described herein based on the supplied pressure provided to the cuff 1640. One or more of a first output signal from the manifold 1612 of the first damping unit 1610, a second output signal from the valve 1622 of the second damping unit 1620, and/or a third output signal from the digital filter 1632 of the third damping unit 1630 can additionally or alternatively be provided to a computing system such as that within pressure controller 1650. In this way, the first output signal indicative of the first amount of damping, the second output signal indicative of the second amount of damping, and the third output signal indicative of the third amount of damping can be utilized not only to assist with blood pressure calculations for a patient but also to derive other hemodynamic parameters of a patient (e.g., heart rate, central venous pressure, temperature, peripheral venous oxygen saturation, blood gas analysis, and the like).

Referring still to FIG. 16, the pressure controller 1650 can be a microprocessor or other computing system, and may be mounted on a printed circuit board (PCB) with the first pressure transducer 1604 and second pressure transducer 1606. Additional details concerning pressure controller 1650 are described herein with reference to FIG. 17. Generally, pressure controller 1650 can be configured to generate a control signal for adjusting one or more parameters of the pressure source provided by the pump 1602 or a supplied pressure provided as an output at a port of the pump system 1601 to cuff 1640. A control signal generated by pressure controller 1650 can be based on at least one of the first amount of damping determined at the first damping unit 1610, the second amount of damping determined at the second damping unit 1620, or the filtered pressure signal which accounts for the third amount of damping determined by the third damping unit 1630.

The first amount of damping determined at the first damping unit 1610 can be greater than the second amount of damping determined at the second damping unit 1620. The first amount of damping determined at the first damping unit 1610 can be greater than the third amount of damping determined at the third damping unit 1630. In other words, the pressure pulsation damping/smoothing or volume ripple reduction implemented by the second damping unit 1620 is more granular than the pressure pulsation damping/smoothing or volume ripple reduction implemented by the first damping unit 1610. Similarly, the pressure pulsation damping/smoothing or volume ripple reduction implemented by the third damping unit 1630 is more granular than the pressure pulsation damping/smoothing or volume ripple reduction implemented by the second damping unit 1620. In some implementations, the first amount of damping determined at the first damping unit 1610 is twice (2×), 5×, 10×, 20×, 100×, or any multiplier between 2× and 100× as much as the second amount of damping determined at the second damping unit 1620.

Display device 1660, similar to display 302 of FIG. 5, can be embedded within or located on a housing that encases the portable blood pressure monitoring system 1600. Display device 1660 can include hardware for displaying a user interface and/or messages to a patient or caretaker of a patient. The user interface or messages provided on display device 1660 can be indicative of a blood pressure measurement or other data calculated by portable blood pressure monitoring system 1600, alerts associated with proper or improper operation of the portable blood pressure monitoring system 1600, etc. By way of example, the display device 1660 can include a display screen, CRT, LCD, plasma screen, touch screen, and/or other suitable display components.

Portable blood pressure monitoring system 1600 can also include one or more additional input/output (I/O) devices 1670. For example, I/O devices 1670 can include one or more input devices that receives user input. For example, the input devices may be a touch-sensitive component (e.g., a touch-sensitive display screen or a touch pad) that is sensitive to the touch of a user input object (e.g., a finger or a stylus). The touch-sensitive component may serve to implement a virtual keyboard. Other example user input components include a microphone, a traditional keyboard, cursor-device, joystick, or other devices by which a user (e.g., a patient, caregiver, or other user) may provide user input. Input devices may be used, for example, to provide input associated with pressure damping, blood pressure measurements, or other inputs associated with the disclosed pump system and blood pressure monitoring systems and methods. I/O devices 1670 can additionally or alternatively include one or more output devices. The output devices can include hardware and/or software for audibly or visually producing content. For instance, the output devices can include one or more display(s), speaker(s), haptic sensor(s), earpiece(s), headset(s), handset(s), etc.

FIG. 17 depicts a block diagram of an example pressure controller 1700 according to example embodiments of the present disclosure. The example pressure controller 1700 includes a computing system 1702 that may be either standalone or communicatively coupled to a network 1750. The pressure controller 1700 of FIG. 17 can correspond to the pressure controller 1650 of FIG. 16. The pressure controller 1700 can be provided on a printed circuit board such as but not limited to PCB 1104 depicted in FIG. 11B.

In some implementations, the computing system 1702 can perform the operations and functions of the various controllers or computing devices described herein. For example, the computing system 1702 can represent a pressure controller, the various components of a pressure controller, and/or other elements described herein and can perform the functions of such elements. The computing system 1702 can include one or more distinct physical computing devices.

The computing system 1702 can include one or more computing devices 1704. The one or more computing devices 1704 can include one or more processors 1706 and a memory device 1708. The one or more processors 1706 can be any suitable processing device (e.g., a processor core, a microprocessor, an ASIC, a FPGA, a controller, a microcontroller, etc.) and can be one processor or a plurality of processors that are operatively connected. The memory device 1708 can include one or more non-transitory computer-readable storage media, such as RAM, ROM, EEPROM, EPROM, one or more memory devices, flash memory devices, etc., and combinations thereof.

The memory device 1708 can store information that can be accessed by the one or more processors 1706. For instance, the memory device 1708 (e.g., one or more non-transitory computer-readable storage mediums, memory devices) can store data 1710 that can be obtained, received, accessed, written, manipulated, created, and/or stored. The data 1710 can include, for instance, data indicative of: one or more pressure signals, one or more electrical signals, damped signals, inputs/output, and/or any other data and/or information as described herein. In some implementations, the computing system 1702 can obtain data from one or more memory device(s) that are remote from the computing system 1702.

The memory device 1708 can also store computer-readable instructions 1712 that can be executed by the one or more processors 1706. The instructions 1712 can be software written in any suitable programming language or can be implemented in hardware. Additionally, or alternatively, the instructions 1712 can be executed in logically and/or virtually separate threads on processor(s) 1706.

For example, the memory device 1708 can store instructions 1712 that when executed by the one or more processors 1706 cause the one or more processors 1706 to perform any of the operations and/or functions described herein, including, for example, the operations and functions of any of the systems/apparatuses described herein, one or more portions of the methods/processes described herein, and/or any other functions or operations.

According to an aspect of the present disclosure, the computing system 1702 can store or include one or more machine-learned models 1716. As examples, the machine-learned models 1716 can be or can otherwise include various machine-learned models such as, for example, neural networks (e.g., deep neural networks), support vector machines, decision trees, ensemble models, k-nearest neighbors models, Bayesian networks, or other types of models including linear models and/or non-linear models. Example neural networks include feed-forward neural networks, recurrent neural networks (e.g., long short-term memory recurrent neural networks), convolutional neural networks, or other forms of neural networks. In some implementations, the computing system 1702 can receive the one or more machine-learned models 1716 over network 1750.

The one or more machine-learned models 1716 can be configured to implement a pressure sensing application, a pressure damping application, a pressure controlling application, a blood pressure calculation application or any other suitable application associated with the systems and devices disclosed herein. In some implementations, the input to the machine learned model(s) 1716 can be a fused input based on signals from multiple sources such as but not limited to signals from the pump, the various damping units, the bladder, or other components of a pump system or a blood pressure monitoring system.

In some implementations, the computing system 1702 can train the machine-learned models 1716 through use of a model trainer 1718. The model trainer 1718 can train the machine-learned models 1716 using one or more training or learning algorithms. One example training technique is backwards propagation of errors. In some implementations, the model trainer 1718 can perform supervised training techniques using a set of labeled training data. In other implementations, the model trainer 1718 can perform unsupervised training techniques using a set of unlabeled training data. The model trainer 1718 can perform a number of generalization techniques to improve the generalization capability of the models being trained. Generalization techniques include weight decays, dropouts, or other techniques.

In particular, the model trainer 1718 can train a machine-learned model 1716 based on a set of training data 1720. The training data 1720 can include, for example, labelled input data and/or fused data indicative of pressure signals, electrical signals, blood pressure measurements or related data, etc. The model trainer 1718 can be implemented in hardware, firmware, and/or software controlling one or more processors.

The computing system 1702 can also include a communication interface 1722 used to communicate with one or more systems or devices, including systems or devices that are remotely located from the computing system 1702. The communication interface 1722 can include any circuits, components, software, etc. for communicating with one or more networks 1750. In some implementations, the communication interface 1722 can include, for example, one or more of a communications controller, receiver, transceiver, transmitter, port, conductors, software and/or hardware for communicating data.

The network(s) 1750 can be any type of network or combination of networks that allows for communication between devices. In some embodiments, the network(s) can include one or more of a local area network, wide area network, the Internet, secure network, cellular network, mesh network, peer-to-peer communication link and/or some combination thereof and can include any number of wired or wireless links. Communication over the network(s) can be accomplished, for instance, via a network interface using any type of protocol, protection scheme, encoding, format, packaging, etc.

FIG. 17 illustrates one example computing system 1702 that can be used to implement aspects of the present disclosure. Other computing systems can be used as well. For example, in some implementations, the computing system 1702 is not connected to other computing systems. The use of computer-based systems allows for a great variety of possible configurations, combinations, and divisions of tasks and functionality between and among components. Computer-implemented operations can be performed on a single component or across multiple components. Computer-implemented tasks and/or operations can be performed sequentially or in parallel. Data and instructions can be stored in a single memory device or across multiple memory devices.

The example routines or methods herein illustrate various implementations of systems described herein. The blocks of the routines illustrate example implementations, and in various other implementations various blocks may be rearranged, and/or rendered optional. Further, various blocks may be omitted from and/or added to the example methods below, and blocks may be moved between the various example methods.

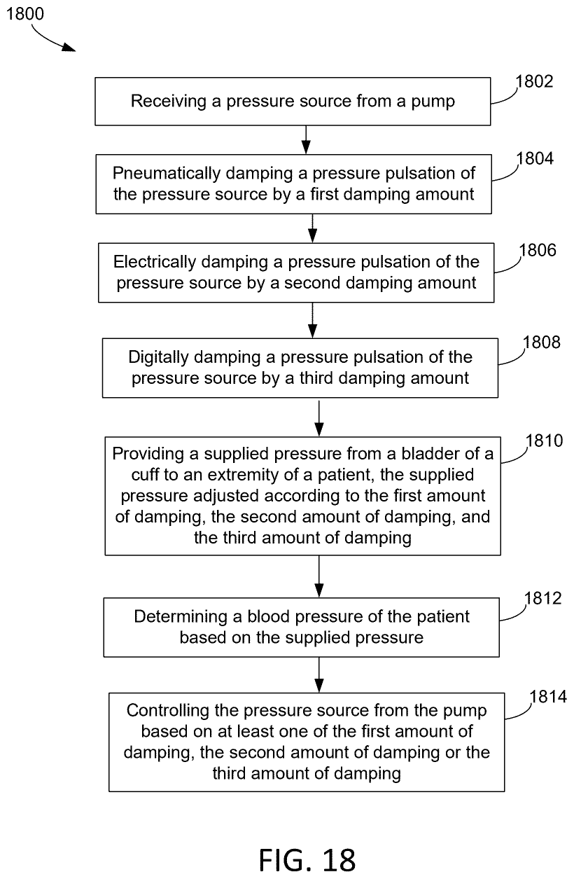

FIG. 18 shows a flowchart of an example method 1800 for controlling pressure in a blood pressure monitoring system. The method 1800 can be performed by one or more systems described herein (e.g., the portable blood pressure monitoring system 1600, the pump system 1601, the pressure controller 1650 or 1700, etc.) and/or a combination thereof. At block 1802, the system can receive a pressure source from a pump, for example pump 1602 of FIG. 16.

At block 1804, the system can pneumatically damp a pressure pulsation of the pressure source received by a first damping amount. The pneumatic damping at block 1804 can be implemented by the first damping unit 1610 of FIG. 16. Volume ripples within the pressure source received from the pump at block 1802 can be reduced by the first damping amount. The pneumatic damping at block 1804 can be implemented by a manifold that constricts channeling of fluid using one or more mechanisms disclosed herein (e.g., multiple pressure chambers, specially configured partitions among connected pressure chambers, tortuous path(s) for fluid flow, etc.) For example, pneumatically damping pressure pulsation of the pressure source can include providing outside air through one or more inlet pressure chambers to an input of the pump. Additionally or alternatively, pneumatically damping pressure pulsation of the pressure source can include providing air from an outlet of the pump through one or more outlet pressure chambers to a port of the housing. Additionally or alternatively, pneumatically damping pressure pulsation of the pressure source can include providing air through a tortuous path comprising a plurality of bends.

At block 1806, the system can electrically damp the pressure pulsation of the pressure source by a second amount of damping. The electric damping at block 1806 can be implemented by the second damping unit 1620 of FIG. 16. Volume ripples within the pressure source received from the pump at block 1802 can be further reduced by the second damping amount. The electric damping at block 1806 can be implemented by a piezoelectric valve configured to implement electric damping by receiving electric signals indicative of pressure within the pump system and controlling a position or state of the valve to increase or decrease fluid flow and corresponding pressure within the pump system. Electric damping at block 1806 can include measuring a cuff pressure within a bladder of a cuff using a first pressure transducer, and controlling a piezoelectric valve to adjust the cuff pressure.

At block 1808, the system can digitally damp the pressure pulsation of the pressure source by a third amount of damping. The digital damping at block 1808 can be implemented by the third damping unit 1630 of FIG. 16. Volume ripples within the pressure source received from the pump at block 1802 can be further reduced by the third damping amount. The digital damping at block 1808 can be implemented by a digital filter, such as a finite impulse response (FIR) filter. The digital damping at block 1808 can be implemented by: receiving a first signal indicative of the supplied pressure as an input; generating a second signal that filters volume ripples in the first signal; and providing the second signal to the pump for controlling the pressure source.

In some examples, the pneumatically damping at block 1804, the electrically damping at block 1806, and the digitally damping at block 1808 are implemented within a housing that stores the pump providing the pressure source at 1802, a manifold configured to implement the pneumatically damping operation at block 1804, a valve configured to implement the electrically damping operation at block 1804, and a filter configured to implement the digitally damping operation at block 1806.

At block 1810, the system can provide a supplied pressure from a bladder of a cuff to an extremity of a patient, the supplied pressure adjusted according to the first amount of damping determined at block 1804, the second amount of damping determined at block 1806, and the third amount of damping determined at block 1808. At block 1812, the system can determine a blood pressure of the patient based on the supplied pressure, e.g., the supplied pressure provided at block 1810. At block 1812, the system can control the pressure source from the pump received at block 1802 based on at least one of the first amount of damping determined at block 1804, the second amount of damping determined at block 1806, or the third amount of damping determined at block 1808. In other examples, the system can additionally or alternatively measure a manifold pressure within the manifold using a second pressure transducer and control the pump to adjust the manifold pressure.

Various means can be configured to perform the methods, operations, and processes described herein. For example, any of the systems and apparatuses (e.g., pump systems, blood pressure monitoring systems, and related circuitry or devices) can include unit(s) and/or other means for performing their operations and functions described herein. In some implementations, one or more of the units may be implemented separately. In some implementations, one or more units may be a part of or included in one or more other units. These means can include processor(s), microprocessor(s), graphics processing unit(s), logic circuit(s), dedicated circuit(s), application-specific integrated circuit(s), programmable array logic, field-programmable gate array(s), controller(s), microcontroller(s), and/or other suitable hardware. The means can also, or alternately, include software control means implemented with a processor or logic circuitry, for example. The means can include or otherwise be able to access memory such as, for example, one or more non-transitory computer-readable storage media, such as random-access memory, read-only memory, electrically erasable programmable read-only memory, erasable programmable read-only memory, flash/other memory device(s), data register(s), database(s), and/or other suitable hardware.

Many variations and modifications may be made to the above-described implementations, the elements of which are to be understood as being among other acceptable examples. All such modifications and variations are intended to be included herein within the scope of this disclosure. The foregoing description details certain implementations. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain implementations include, while other implementations do not include, certain features, elements, and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or “at least one of X, Y, or Z,” unless specifically stated otherwise, is to be understood with the context as used in general to convey that an item, term, etc. may be either X, Y, or Z, or a combination thereof.

For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain implementations require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a computer comprising one or more processors should not be interpreted as excluding other computer components, and may possibly include such components as memory, input/output devices, and/or network interfaces, among others.

While the above detailed description has shown, described, and pointed out novel features as applied to various implementations, it may be understood that various omissions, substitutions, and changes in the form and details of the devices or processes illustrated may be made without departing from the spirit of the disclosure. As may be recognized, certain implementations of the inventions described herein may be embodied within a form that does not provide all of the features and benefits set forth herein, as some features may be used or practiced separately from others. Each of the disclosed aspects and examples of the present disclosure may be considered individually or in combination with other aspects, examples, and variations of the disclosure. The headings used herein are merely provided to enhance readability and are not intended to limit the scope of the embodiments disclosed in a particular section to the features or elements disclosed in that section. The features or elements from one embodiment of the disclosure can be employed by other embodiments of the disclosure. For example, features described in one figure may be used in conjunction with embodiments illustrated in other figures. The foregoing description and examples have been set forth merely to illustrate the disclosure and are not intended as being limiting. The scope of certain inventions disclosed herein is indicated by the appended claims rather than by the foregoing description. All changes which come within the meaning and range of equivalency of the claims are to be embraced within their scope.

Some example embodiments are included below for illustrative purposes. These examples should not be viewed as limiting.

In Example 1, a pump system comprising: a tortuous path; and a pump which is connected to an outlet of the tortuous path, wherein an inlet of the tortuous path is configured to receive outside air such that the received air travels through the tortuous path prior to entering the pump.

In Example 2, the pump system of Example 1, wherein the tortuous path has no moving element.

In Example 3, the pump system of any of Examples 1-2, further comprising a volume accumulator attached to the pump which has a volume equal to a stroke volume of the pump.

In Example 4, the pump system of Example 3, further comprising an orifice attached to the pump which receives air from the volume accumulator and wherein a diameter of the orifice is 0.1-times the stroke volume of the pump and the volume of the accumulator.

In Example 5, the pump system of any of Examples 1-4, wherein the pump comprises one or more active damping units which measure pressure of volume ripples from the pump and control a flow of air.

In Example 6, the pump system of Example 5, wherein the one or more active damping units comprises a bellow or a poppet.

In Example 7, the pump system of any of Examples 1-6, wherein the tortuous path is elongated and snakes back and forth laterally.

In Example 8, the pump system of any of Examples 1-7, wherein the tortuous path has a circular cross-section with a diameter and a path length.

In Example 9, the pump system of any of Examples 1-8, wherein a path length of the tortuous path is 150-times to 200-times longer than a diameter of the tortuous path.

In Example 10, the pump system of any of Examples 1-9, wherein a path length of the tortuous path is between 18 inches and 30 inches.

In Example 11, the pump system of any of Examples 1-10, further comprising a printed circuit board which controls the pump.

In Example 12, the pump system of Example 11, further comprising a battery which powers the printed circuit board.

In Example 13, the pump system of Example 12, further comprising a housing and a baseplate which cooperate to house the pump, the printed circuit board, and the battery.

In Example 14, a non-invasive blood pressure monitoring system comprising: a pump system comprising: a tortuous path; and a pump which is connected to an outlet of the tortuous path, wherein an inlet of the tortuous path is configured to receive outside air such that the air travels through the tortuous path prior to entering the pump, and a cuff comprising a bladder connected to an output of the pump system, wherein the bladder is configured to apply pressure to non-invasively measure blood pressure.

In Example 15, a method of damping pressure pulsations from a pump, the method comprising: providing air through an inlet of a tortuous path; and pumping the provided air by a pump connected to an outlet of the tortuous path, wherein the inlet of the tortuous path is configured to receive outside air such that the air travels through the tortuous path prior to entering the pump.

In Example 16, the method of Example 15, wherein the inlet of the tortuous path provides reduction of noise created by the pump by having no moving element within the tortuous path.

In Example 17, the method of any of Examples 14-15, wherein the tortuous path has no moving element.

In Example 18, the method of any of Examples 14-17, wherein the tortuous path is elongated and snakes back and forth laterally.

In Example 19, the method of any of Examples 14-18, wherein the tortuous path has a circular cross-section with a diameter and a path length.

In Example 20, the method of any of Examples 14-19, wherein a path length of the tortuous path is 150-times to 200-times longer than a diameter of the tortuous path.

In Example 21, a pump system comprising: a pump including an inlet and an outlet; inlet pressure chambers connected to the inlet, wherein the inlet pressure chambers are connected in series to such that a last inlet pressure chamber is connected to the inlet of the pump and a beginning inlet pressure chamber is connected to outside air; outlet pressure chambers connected to the outlet, wherein each of the outlet pressure chambers are connected in series such that a beginning outlet pressure chamber is connected to the outlet of the pump and a last outlet pressure chamber is connected to a port.

In Example 22, the pump system of Example 21, wherein the inlet pressure chambers are connected in series through one or more passages.

In Example 23, the pump system of Example 22, wherein each of the one or more passages are formed by a first opening which includes an orifice, a second opening, and a channel, and wherein air is sucked from a first inlet pressure chamber through the orifice, through the channel, through the second opening, and into a second inlet pressure chamber.

In Example 24, the pump system of Example 23, wherein the orifice is a thin disk material including a small hole.

In Example 25, the pump system of Example 24, wherein the first opening and the second opening are formed in a gasket and the thin disk material of the orifice is thinner than the gasket.

In Example 26, the pump system of any of Examples 21-25, wherein each of the one or more passages are formed by a first opening, a second opening which includes an orifice, and a channel, wherein air is pushed through a first inlet pressure chamber, through the first opening, through the channel, through the orifice, and into a second inlet pressure chamber.

In Example 27, the pump system of any of Examples 21-25, wherein each of the one or more passages are formed by a first opening which includes a first orifice, a second opening which includes a second orifice, and a channel, wherein air is pushed through a first inlet pressure chamber, through the first orifice, through the channel, through the second orifice, and into a second inlet pressure chamber.

In Example 28, the pump system of any of Examples 21-27, wherein the inlet pressure chambers comprise three pressure chambers.

In Example 29, the pump system of any of Examples 21-28, wherein the outlet pressure chambers are connected in series through one or more passages.

In Example 30, the pump system of Example 29, wherein each of the one or more passages are formed by a first opening which includes an orifice, a second opening, and a channel, and wherein air is pushed through a first outlet pressure chamber, through the orifice, through the channel, through the second opening, and into a second outlet pressure chamber.

In Example 31, the pump system of Example 30, wherein the orifice is a thin disk material including a small hole.

In Example 32, the pump system of Example 31, wherein the first opening and the second opening are formed in a gasket and the thin disk material of the orifice is thinner than the gasket.

In Example 33, the pump system of any of Examples 29-32, wherein each of the one or more passages are formed by a first opening, a second opening which includes an orifice, and a channel, wherein air is pushed through a first outlet pressure chamber, through the first opening, through the channel, through the orifice, and into a second outlet pressure chamber.

In Example 34, the pump system of any of Examples 29-33, wherein each of the one or more passages are formed by a first opening which includes a first orifice, a second opening which includes a second orifice, and a channel, wherein air is pushed through a first outlet pressure chamber, through the first orifice, through the channel, through the second orifice, and into a second outlet pressure chamber.

In Example 35, the pump system of any of Examples 21-34, wherein the outlet pressure chambers comprise four pressure chambers.

In Example 36, the pump system of any of Examples 21-35, wherein the last outlet pressure chamber is connected to the port through a piezoelectric valve.

In Example 37, the pump system of Example 36, wherein the piezoelectric valve is switchable such that, in a first setting, the last outlet pressure chamber is connected to the port and, in a second setting, both the last outlet pressure chamber and the port are connected to outside air.

In Example 38, the pump system of any of Examples 36-37, further comprising a manifold including the inlet pressure chambers and the outlet pressure chambers and a printed circuit board including a first pressure transducer and a second pressure transducer, wherein the printed circuit board is mounted on the manifold with the piezoelectric valve positioned between the printed circuit board and the manifold, and wherein the piezoelectric valve is positioned between the first pressure transducer and the second pressure transducer.

In Example 39, the pump system of Example 38, wherein the first pressure transducer is configured to measure the pressure at the port and the second pressure transducer is configured to measure the pressure before the piezoelectric valve.

In Example 40, a non-invasive blood pressure monitoring system comprising: a pump system comprising: a pump including an inlet and an outlet; inlet pressure chambers connected to the inlet, wherein the inlet pressure chambers are connected in series to such that a last inlet pressure chamber is connected to the inlet of the pump and a first inlet pressure chamber is connected to outside air; outlet pressure chambers connected to the outlet, wherein each of the outlet pressure chambers are connected in series such that a first outlet pressure chamber is connected to the outlet of the pump and a last outlet pressure chamber is connected to a port, and a cuff comprising a bladder connected to the port, wherein the bladder is configured to apply pressure to non-invasively measure blood pressure.

In Example 41, a portable blood pressure monitoring system comprising: a pump including an inlet and an outlet, the pump configured to provide a pressure source; a first damping unit coupled to the pump, the first damping unit comprising a manifold configured to damp pressure pulsation of the pressure source by a first amount of damping; and a second damping unit coupled to an output of the first damping unit, the second damping unit comprising a valve configured to damp pressure pulsation of the pressure source by a second amount of damping; and a cuff comprising a bladder connected to an output of the second damping unit, wherein the bladder is configured to provide a supplied pressure to an extremity of a patient to non-invasively measure blood pressure of the patient, the supplied pressure adjusted according to the first amount of damping and the second amount of damping.

In Example 42, the portable blood pressure monitoring system of Example 41, comprising a housing configured to encase the pump, the first damping unit, and the second damping unit.

In Example 43, the portable blood pressure monitoring system of Example 42, wherein the cuff is positioned on a surface of the housing.

In Example 44, the portable blood pressure monitoring system of Example 43, wherein the extremity of the patient comprises a finger of the patient, and wherein the bladder is configured to receive the finger of the patient while a hand of the patient is positioned on the surface of the housing.

In Example 45, the portable blood pressure monitoring system of any of Examples 41-45, wherein: the first damping unit comprises one or more pressure chambers connected to an outlet of the pump and to a port; and the valve of the second damping unit comprises a piezoelectric valve configured to vent pressure at the cuff.

In Example 46, the portable blood pressure monitoring system of Example 45, wherein the piezoelectric valve is switchable such that, in a first setting, the one or more pressure chambers are connected to the port and, in a second setting, both the one or more pressure chambers and the port are connected to outside air.

In Example 47, the portable blood pressure monitoring system of any of Examples 45-46, wherein: the second damping unit comprises a first pressure transducer and a second pressure transducer; the first pressure transducer is configured to measure pressure before the cuff; and the second pressure transducer is configured to measure pressure before the piezoelectric valve.

In Example 48, the portable blood pressure monitoring system of Example 47, wherein the piezoelectric valve is positioned between the first pressure transducer and the second pressure transducer.

In Example 49, the portable blood pressure monitoring system of any of Examples 47-48, comprising a controller configured to: receive an output signal from the first pressure transducer indicative of a cuff pressure of the bladder of the cuff and an output signal from the second pressure transducer indicative of a manifold pressure of the manifold; generate a pump control signal for adjusting one or more parameters of the pressure source provided by pump; and generate a cuff control signal for adjusting one or more parameters of the supplied pressure provided by the bladder of the cuff.

In Example 50, the portable blood pressure monitoring system of any of Examples 41-49, further comprising a filter configured to: receive a first signal indicative of the supplied pressure as an input; generate a second signal that filters volume ripples in the first signal; and provide the second signal to the pump for controlling the pressure source.

In Example 51, the portable blood pressure monitoring system of Example 50, wherein the filter comprises a finite impulse response (FIR) filter.

In Example 52, the portable blood pressure monitoring system of any of Examples 41-51, wherein the manifold comprises one or more pressure chambers connected in series, at least one pressure chamber of the one or more pressure chambers coupling the outlet of the pump to a port.

In Example 53, the portable blood pressure monitoring system of Example 52, wherein a first pressure chamber and a second pressure chamber of the one or more pressure chambers are: (i) separated by a partition, and (ii) connected in series through a first opening, a channel, and a second opening provided at the partition.

In Example 54, the portable blood pressure monitoring system of any of Examples 41-53, wherein the manifold comprises a tortuous path including a plurality of bends through which air forming the pressure source passes through prior to entering the pump.

In Example 55, a method of controlling pressure in a non-invasive blood pressure monitoring system, comprising: receiving a pressure source from a pump; pneumatically damping a pressure pulsation of the pressure source by a first amount of damping; electrically damping the pressure pulsation of the pressure source by a second amount of damping; digitally damping the pressure pulsation of the pressure source by a third amount of damping; providing a supplied pressure from a bladder of a cuff to an extremity of a patient, the supplied pressure adjusted according to the first amount of damping, the second amount of damping, and the third amount of damping; and determining a blood pressure of the patient based on the supplied pressure.

In Example 56, the method of Example 55, wherein the pneumatically damping, the electrically damping, and the digitally damping operations are implemented within a housing that stores the pump, a manifold configured to implement the pneumatically damping operation, a valve configured to implement the electrically damping operation, and a filter configured to implement the digitally damping operation.

In Example 57, the method of Example 56, wherein pneumatically damping the pressure pulsation of the pressure source comprises providing outside air through one or more inlet pressure chambers to an input of the pump.

In Example 58, the method of any of Examples 56-57, wherein pneumatically damping the pressure pulsation of the pressure source comprises providing air from an outlet of the pump through one or more outlet pressure chambers to a port of the housing.

In Example 59, the method of any of Examples 56-58, wherein pneumatically damping the pressure pulsation of the pressure source comprises providing air through a tortuous path comprising a plurality of bends.

In Example 60, the method of any of Examples 55-59, wherein digitally damping the pressure pulsation of the pressure source comprises providing the pressure source to a finite impulse response (FIR) filter.

In Example 61, the method of any of Examples 56-60, wherein electrically damping the pressure pulsation of the pressure source comprises providing a first pressure transducer, a second pressure transducer, and a piezoelectric valve positioned between the first pressure transducer and the second pressure transducer.

In Example 62, the method of Example 61, comprising: measuring a cuff pressure within the bladder of the cuff using the first pressure transducer; and controlling the piezoelectric valve to adjust the cuff pressure.

In Example 63, the method of any of Examples 61-62, comprising: measuring a manifold pressure within the manifold using the second pressure transducer; and controlling the pump to adjust the manifold pressure.

In Example 64, the method of any of Examples 61-63, comprising: switching the piezoelectric valve between a first setting configured to connect one or more pressure chambers between the pump and the bladder of the cuff and a second setting configured to connect the one or more pressure chambers and the bladder of the cuff to outside air.

In Example 65, the method of any of Examples 55-64, comprising controlling the pressure source from the pump based on at least one of the first amount of damping, the second amount of damping, or the third amount of damping.

In Example 66, the method of any of Examples 61-65, wherein electrically damping the pressure pulsation of the pressure source by a second amount of damping comprises: receiving an output signal from a first pressure transducer indicative of a cuff pressure of the bladder of the cuff and an output signal from a second pressure transducer indicative of a manifold pressure of the manifold; generating a pump control signal for adjusting one or more parameters of the pressure source provided by pump; and generating a cuff control signal for adjusting one or more parameters of the supplied pressure provided by the bladder of the cuff.

In Example 67, the method of any of Examples 55-66, wherein digitally damping the pressure pulsation of the pressure source by a third amount of damping comprises: receiving a first signal indicative of the supplied pressure as an input; generating a second signal that filters volume ripples in the first signal; and providing the second signal to the pump for controlling the pressure source.

In Example 68, a pump system with integrated pressure control features and a port, the pump system comprising: a pump configured to provide a pressure source; a first damping unit coupled to the pump, the first damping unit comprising a manifold configured to damp pressure pulsation of the pressure source by a first amount of damping; a second damping unit coupled to an output of the first damping unit, the second damping unit comprising a valve configured to damp pressure pulsation of the pressure source by a second amount of damping; a filter coupled to an output of the second damping unit and configured to generate a filtered pressure signal characterized by a further reduction in pressure pulsation; and a controller configured to generate a control signal for adjusting one or more parameters of the pressure source provided by the pump or a supplied pressure provided as an output at a port of the pump system, the control signal based on at least one of the first amount of damping, the second amount of damping, or the filtered pressure signal.

In Example 69, the pump system of Example 68, wherein: the first damping unit comprises one or more pressure chambers connected to an outlet of the pump and to the port; and the valve of the second damping unit comprises a piezoelectric valve configured to vent pressure at the port.

In Example 70, the pump system of Example 69, wherein the piezoelectric valve is switchable such that, in a first setting, the one or more pressure chambers are connected to the port and, in a second setting, both the one or more pressure chambers and the port are connected to outside air.

In Example 71, the pump system of any of Examples 69-70, wherein: the second damping unit comprises a first pressure transducer and a second pressure transducer; the first pressure transducer is configured to measure pressure at the port; and the second pressure transducer is configured to measure pressure before the piezoelectric valve.

In Example 72. the pump system of Example 71, wherein the piezoelectric valve is positioned between the first pressure transducer and the second pressure transducer.

In Example 73, the pump system of any of Examples 71-72, wherein the second damping unit comprises a printed circuit board, wherein the first pressure transducer and the second pressure transducer are mounted on the printed circuit board.

In Example 74, the pump system of Example 73, wherein the printed circuit board is mounted on the manifold with the piezoelectric valve positioned between the printed circuit board and the manifold.

In Example 75, the pump system of any of Examples 68-74, wherein the manifold comprises one or more pressure chambers connected in series, at least one pressure chamber of the one or more pressure chambers coupling an outlet of the pump to the port.

In Example 76, the pump system of Example 75, wherein a first pressure chamber and a second pressure chamber of the one or more pressure chambers are: (i) separated by a partition, and (ii) connected in series through a first opening, a channel, and a second opening provided at the partition.

In Example 77, the pump system of any of Examples 68-76, wherein the manifold comprises a tortuous path including a plurality of bends through which air forming the pressure source passes through prior to entering the pump.

In Example 78, the pump system of any of Examples 68-77, wherein the filter comprises a finite impulse response (FIR) filter.

In Example 79, the pump system of any of Examples 68-78, wherein the manifold comprises one or more inlet pressure chambers connected to an inlet of the pump, wherein the inlet pressure chambers are connected in series to such that a last inlet pressure chamber is connected to the inlet of the pump and a first inlet pressure chamber is connected to outside air.

In Example 80, the pump system of any of Examples 68-79, wherein the manifold comprises one or more outlet pressure chambers connected to an outlet of the pump, wherein each of the outlet pressure chambers are connected in series such that a first outlet pressure chamber is connected to the outlet of the pump and a last outlet pressure chamber is connected to the port.

What is claimed is:

1. A portable blood pressure monitoring system comprising:
a pump including an inlet and an outlet, the pump configured to provide a pressure source;
a first damping unit coupled to the pump, the first damping unit comprising a manifold configured to damp pressure pulsation of the pressure source by a first amount of damping; and
a second damping unit coupled to an output of the first damping unit, the second damping unit comprising a valve configured to damp pressure pulsation of the pressure source by a second amount of damping;
a third damping unit coupled to an output of the second damping unit, the third damping unit comprising a filter configured to damp pressure pulsation of the pressure source by a third amount of damping; and
a cuff comprising a bladder connected to an output of the second damping unit, wherein the bladder is configured to provide a supplied pressure to an extremity of a patient to non-invasively measure blood pressure of the patient, the supplied pressure adjusted according to the first amount of damping, the second amount of damping, and the third amount of damping.

2. The portable blood pressure monitoring system of claim 1, comprising a housing configured to encase the pump, the first damping unit, the second damping unit, and the third damping unit.

3. The portable blood pressure monitoring system of claim 2, wherein the cuff is positioned on a surface of the housing.

4. The portable blood pressure monitoring system of claim 1, wherein:
the first damping unit comprises one or more pressure chambers connected to an outlet of the pump and to a port; and
the valve of the second damping unit comprises a piezoelectric valve configured to vent pressure at the cuff.

5. The portable blood pressure monitoring system of claim 4, wherein the piezoelectric valve is switchable such that, in a first setting, the one or more pressure chambers are connected to the port and, in a second setting, both the one or more pressure chambers and the port are connected to outside air.

6. The portable blood pressure monitoring system of claim 5, comprising:
a first pressure transducer configured to measure pressure before the cuff; and
a second pressure transducer configured to measure pressure before the piezoelectric valve.

7. The portable blood pressure monitoring system of claim 6, comprising a controller configured to:
receive an output signal from the first pressure transducer indicative of a cuff pressure of the bladder of the cuff and an output signal from the second pressure transducer indicative of a manifold pressure of the manifold;
generate a pump control signal for adjusting one or more parameters of the pressure source provided by pump; and
generate a cuff control signal for adjusting one or more parameters of the supplied pressure provided by the bladder of the cuff.

8. The portable blood pressure monitoring system of claim 1, wherein the filter is configured to:
receive a first signal indicative of the supplied pressure as an input;
generate a second signal that filters volume ripples in the first signal; and
provide the second signal for determining the blood pressure of the patient.

9. The portable blood pressure monitoring system of claim 1, wherein the filter comprises a finite impulse response (FIR) filter.

10. The portable blood pressure monitoring system of claim 1, wherein the manifold comprises one or more pressure chambers connected in series, at least one pressure chamber of the one or more pressure chambers coupling the outlet of the pump to a port.

11. The portable blood pressure monitoring system of claim 10, wherein a first pressure chamber and a second pressure chamber of the one or more pressure chambers are: (i) separated by a partition, and (ii) connected in series through a first opening, a channel, and a second opening provided at the partition.

12. The portable blood pressure monitoring system of claim 1, wherein the manifold comprises a tortuous path including a plurality of bends through which air forming the pressure source passes through prior to entering the pump.

13. A method of controlling pressure in a blood pressure monitoring system, the method comprising:
receiving a pressure source from a pump;
pneumatically damping a pressure pulsation of the pressure source by a first amount of damping;
electrically damping the pressure pulsation of the pressure source by a second amount of damping;
digitally damping the pressure pulsation of the pressure source by a third amount of damping;
providing a supplied pressure from a bladder of a cuff to an extremity of a patient, the supplied pressure adjusted according to the first amount of damping, the second amount of damping, and the third amount of damping; and
determining a blood pressure of the patient based on the supplied pressure.

14. The method of claim 13, wherein the pneumatically damping, the electrically damping, and the digitally damping operations are implemented within a housing that stores the pump, a manifold configured to implement the pneumatically damping operation, a valve configured to implement the electrically damping operation, and a filter configured to implement the digitally damping operation.

15. The method of claim 14, wherein pneumatically damping the pressure pulsation of the pressure source comprises providing outside air through one or more inlet pressure chambers to an input of the pump.

16. The method of claim 15, wherein pneumatically damping the pressure pulsation of the pressure source comprises providing air from an outlet of the pump through one or more outlet pressure chambers to a port of the housing.

17. The method of claim 13, wherein pneumatically damping the pressure pulsation of the pressure source comprises providing air through a tortuous path comprising a plurality of bends.

18. The method of claim 13, wherein digitally damping the pressure pulsation of the pressure source comprises providing the pressure source to a finite impulse response (FIR) filter.

19. The method of claim 14, wherein electrically damping the pressure pulsation of the pressure source comprises:
measuring a cuff pressure within the bladder of the cuff using a first pressure transducer;
and controlling a piezoelectric valve to adjust the cuff pressure.

20. The method of claim 19, comprising:
measuring a manifold pressure within the manifold using a second pressure transducer; and
controlling the pump to adjust the manifold pressure.

21. The method of claim 13, comprising controlling the pressure source from the pump based on at least one of the first amount of damping, the second amount of damping, or the third amount of damping.

22. The method of claim 13, wherein digitally damping the pressure pulsation of the pressure source by a third amount of damping comprises:
receiving a first signal indicative of the supplied pressure as an input;
generating a second signal that filters volume ripples in the first signal; and
provide the second signal for determining the blood pressure of the patient.

23. A pump system with integrated pressure control features and a port, the pump system comprising:
a pump configured to provide a pressure source;
a first damping unit coupled to the pump, the first damping unit comprising a manifold configured to damp pressure pulsation of the pressure source by a first amount of damping;
a second damping unit coupled to an output of the first damping unit, the second damping unit comprising a valve configured to damp pressure pulsation of the pressure source by a second amount of damping;
a filter coupled to an output of the second damping unit and configured to generate a filtered pressure signal characterized by a further reduction in pressure pulsation; and
a controller configured to generate a control signal for adjusting one or more parameters of the pressure source provided by the pump or a supplied pressure provided as an output at a port of the pump system, the control signal based on at least one of the first amount of damping, the second amount of damping, or the filtered pressure signal.

24. The pump system of claim 23, wherein:
the first damping unit comprises one or more pressure chambers connected to an outlet of the pump and to the port; and
the valve of the second damping unit comprises a piezoelectric valve configured to vent pressure at the port.

25. The pump system of claim 24, comprising:
a first pressure transducer configured to measure pressure at the port; and
a second pressure transducer configured to measure pressure before the piezoelectric valve.

26. The pump system of claim 25, wherein:
the first pressure transducer and the second pressure transducer are mounted on a printed circuit board provided on the manifold; and
the piezoelectric valve is positioned between the printed circuit board and the manifold.

27. The pump system of claim 23, wherein the manifold comprises one or more pressure chambers connected in series, at least one pressure chamber of the one or more pressure chambers coupling an outlet of the pump to the port.

28. The pump system of claim 27, wherein a first pressure chamber and a second pressure chamber of the one or more pressure chambers are: (i) separated by a partition, and (ii) connected in series through a first opening, a channel, and a second opening provided at the partition.

29. The pump system of claim 23, wherein the manifold comprises a tortuous path including a plurality of bends through which air forming the pressure source passes through prior to entering the pump.

30. The pump system of claim 23, wherein the filter comprises a finite impulse response (FIR) filter.
